(19) United States
(12) Patent Application Publication
(10) Pub. No.: US 2015/0313801 A1
(43) Pub. Date: Nov. 5, 2015

(54) DETECTION OF CARIOUS DENTIN TISSUE AND REMOVAL THEREOF BY MEANS OF A DENTAL INSTRUMENT

(71) Applicant: RLS GLOBAL AB, Gothenburg (SE)

(72) Inventors: Ulrica ALMHÖJD, Bohus-björkö (SE); Åke NILSSON, Gothenburg (SE); Fredrik FLORBERGER, Torslanda (SE); Kenth HANSSEN, Torslanda (SE)

(21) Appl. No.: 14/647,495

(22) PCT Filed: Nov. 29, 2013
(86) PCT No.: PCT/EP2013/075080

§ 371 Date: May 27, 2015

Related U.S. Application Data
(60) Provisional application No. 61/731,571, filed on Nov. 30, 2012.

(30) Foreign Application Priority Data
Nov. 30, 2012 (SE) 1251362-8
Apr. 12, 2013 (SE) 1350468-3

(51) Int. Cl. A61K 6/00; A61C 5/02; G01N 23/225; A61B 5/00; G01N 33/483; G01N 21/35; A61C 3/02; A61C 19/06

(52) U.S. Cl. CPC A61K 6/0061; A61C 3/02; A61C 5/023; A61C 19/063; A61B 5/4547; G01N 33/4833; G01N 21/35; G01N 23/2258; A61K 6/002; G01N 2021/3595

(57) ABSTRACT

The application relates to a method for testing a dental instrument comprising the steps of: a) subjecting healthy dentin tissue free from smear layer(s) to treatment with a dental instrument, thereby yielding treated dentin tissue, b) addition of a composition comprising one or more hydrazine derivative(s) to the treated dentin tissue obtained in step a), c) examining if the treated dentin tissue in step b) is stained by the one or more hydrazine derivative(s), and d) determining whether or not the treated dentin tissue contains a smear layer, wherein said one or more hydrazine derivatives is a compound of formula (I) RNHNH₂ wherein R is a chemical group containing a chromophore or forming a chromophore with NHNH₂. Further, the application relates to a kit of parts for detection and removal of carious dentin tissue and to a method for detection and removal of carious dentin tissue.

### TECHNICAL FIELD

The present invention concerns dental caries. In particular, the present invention concerns detection of carious dentin tissue using hydrazine derivative(s), and selective removal of detected carious tissue.

### BACKGROUND

In the treatment of carious lesions the major part of the carious tissue can be examined by judging the hardness of the affected surface using tactile instruments (so-called probing) and/or by visually observing the well-known discoloration known as Maillard products. However, the caries affected surface near the healthy dentin, which is called the transparent zone, is not discoloured.

For early stage caries where the caries only affects the enamel of the tooth different therapies are used. In one therapy one removes the caries affected tissue using mechanical means such as dental drill tools. Another therapy uses a preventive methodology such as fluoridation.

Initial caries, i.e. caries affecting only the enamel of the tooth, is normally detectable for the dentist as a diminished translucence in areas of caries, and can be observed as an opaque area due to the porous enamel. However, detection of caries in the inter proximal area between teeth requires radiographic analysis.

In all situations it might not be possible to remove all carious tissue. For instance, this may be the case when the carious lesions have progressed into the dentin of the tooth. Some carious dentin tissue may then still remain and this carious tissue may become locked in underneath a filling used to repair the tooth. This will certainly cause further development of carious dentin tissue, which can develop unseen due to the fact that it is hidden underneath the filling. When the affected individual becomes aware of the carious dentin tissue it might be hard to save the tooth, and extraction or root filling may be needed.

This problem can of course be handled by excavating healthier dentin tissue from the tooth; i.e. healthy dentin tissue will be removed in addition to the carious dentin tissue. However, this will lead to a weakening of the tooth strength (i.e. less crystals, hydroxyapatite, and less proteins, respectively), and the crown walls may become too thin. Further this will lead to a decrease in the tooth re-mineralisation process since tissue is replaced by a synthetic material like a composite. Taking away tissue affects the whole tooth, both the inorganic and the organic parts.

US 2007/0287122 describes a method of treating residual caries comprising the steps of a) flushing carious tissue with a stain chosen to readily absorb energy from a chosen laser source, b) allowing some of the stain to be absorbed by the carious tissue; c) rinsing the area, leaving the absorbed stain in the carious tissue; and d) removing carious tissue with ablation by laser of a complimentary wavelength and subsequent etching of remaining inorganic tissue using an acid.

US 2010/0119987 encompasses the disclosure of US 2007/0287122, and further disclose test results on a piece of pork loin. It is mentioned that the method may be used in caries treatment.

US 2010/0145191 discloses a method for staining a selected tissue with a dye, stain or pigment that is attenuated to absorb the energy from a radiant energy source.

WO 2008/048170 discloses a method for determining the presence of carious dentin tissue by means of infrared spectroscopy, which is based on the finding that ester groups are present in carious tissue in contrast to healthy dentin tissue that lacks ester groups.

WO 98/020838 describes a preparation for chemical-mechanical treatment of carious tissue in the form of a two-component caries-dissolving liquid including a coloring agent such as Erythrosin.

WO 2007/123880 discloses a method and a kit for early stage caries detection wherein the early dental caries may be detected by binding an optically detectable probe such as Hylight Fluor to the enamel caries an using an optical device for detection. Nothing is mentioned about detection of dental caries that has progressed into the dentin tissue; i.e. carious dentin tissue.

It is well known in the art that healthy dentin tissue comprises amide groups. This is described in, for instance, Digestion and Nutrition: In Zoology by Dorit, ILR. and Walker, W. F, Saunders College, page 247 as well as in Primary Structure Determination In Biochemistry by Voet, D. and Voet, G. E., 2^{nd} edition, John Wiley & Sons, Inc. NY, at page 156.

PCT/EP2012/061494 discloses a kit of parts for labelling and removal of carious dentin tissue comprising: (i) one or more compounds being a hydrazine derivative, and (ii) means for chemical treatment of carious dentin tissue. The hydrazine derivative labels carious dentin tissue selectively and substantially irreversibly in the presence of healthy dentin tissue and allows for removal of the carious dentin tissue with the means for chemical treatment. However, selective detection and removal using hydrazine derivatives and a dentist's drill was unsuccessful.

US2007/0248935 discloses a dental drill having a head provided with cutting edges, the head having an at least rounded basic shape, wherein two of the cutting edges merge into a transitional cutting edge at the front side. The dental drill is made of a ceramic material and that the cutting edge chipping is between 2.5 and 7 μm and the cutting edge base has a radius between 0.03 and 0.12 mm. The dental drill is said to be particularly suited for removing diseased dentin from cavities in case of a cavity preparation, wherein diseased dentin is removed, however, sane dentin is preserved.

US 2009/0208902 discloses a dental drill comprising a head provided with cutting edges, which has an at least rounded basic shape, wherein two of the cutting edges at the front side merge into a transitional cutting edge. The dental drill is made of a plastics material which has a hardness between 60 and 250 Knoop. The dental drill is said to be particularly suited for removing diseased dentin from cavities in case of a cavity preparation, wherein diseased dentin is removed, however, sane dentin is preserved.

### SUMMARY OF THE INVENTION

It is an object of the present invention to increase the versatility of hydrazine derivatives in dental treatment such as detection of carious dentin tissue.

The object is wholly or partially achieved by a method according to appended claim 1, a kit of parts according to appended claim 9, a method according to appended claim 19. Embodiments are set forth in the appended dependent claims and in the following description and examples. Advantages and of the aspects and embodiments of the present invention will be further described in the detailed description. Definitions of terms used herein will also be presented in the detailed description

The present invention is based on the insight that a composition comprising one or more hydrazine derivative(s) may be used for detection and/or labelling of carious dentin tissue in dentin tissue free of smear layer(s). The absence of smear layer(s) is important, since the inventors of the present invention have found that hydrazine derivative(s) stain(s) smear layer(s) making it difficult to judge if the staining is due to the presence of carious dentin tissue or smear layer(s). The thus labelled carious dentin tissue may be removed by mechanical means such as a dental drill thereby yielding healthy dentin tissue. Surprisingly, the inventors have found that some dental instruments such as some dental drills do not yield dentin tissue comprising smear layer(s) upon treatment of the dentin tissue having or lacking smear layer(s) with said instrument, and therefore allow for checking the presence or absence of possibly remaining carious dentin tissue.

Accordingly, in a first aspect there is provided a method for testing a dental instrument. The method comprises the steps of:
a) subjecting healthy dentin tissue free from smear layer(s) of an extracted healthy tooth to treatment with a dental instrument, thereby yielding a drilled treated dentin tissue,
b) addition of a composition comprising one or more hydrazine derivative(s) to the drilled dentin tissue obtained in step a), and
c) examining if the treated dentin tissue by the dental instrument in step b) is stained by the one or more hydrazine derivative(s), and
d) determining whether or not the treated dentin tissue contains a smear layer, wherein a stained dentin tissue in step c) indicates the presence of a smear layer, and wherein a lack of stained dentin tissue in step c) indicates the absence of a smear layer

Said one or more hydrazine derivatives is a compound of formula (I)

RNHNH₂ (I)

wherein R is a chemical group containing a chromophore or forming a chromophore with NHNH₂.

The healthy tooth with dentin tissue free from smear layer(s) may be obtained by subjecting the healthy tooth and the dentin tissue thereof to acid etching or to treatment with a preparation as described further herein, the preparation containing an active, caries-dissolving two-component liquid in the form of a first active, caries-dissolving component and the a second component which reduces the aggressiveness of the active component to mucous membranes, and a gel substance. The preparation may, for example, be Carisolv®, Perioplus® or Perisolv.

The method may further comprise one or more rinsing steps, wherein rinsing is performed with water, brine or an aqueous pharmacologically acceptable solution.

The preparation may be removed from the tooth or teeth by a fluid flow, such as an air flow, and/or by a powder-like blasting, wherein healthy dentin tissue free from smear layer(s) is obtained (also see hereinbelow). This may be provided with a flow apparatus as described hereinbelow. Such flow apparatus may constitute the dental instruments as such.

The examination in step (c) may take place using an instrument operating between 10 and 500 nm, preferably between 200 and 500 nm, and more preferably between 10 and 400 nm, or any other operating wavelength as described herein.

The dental instrument may be a dental drill as described herein, wherein the treatment with the dental instrument in step a) is provided by drilling with the dental drill thereby yielding drilled dentin tissue.

The dental instrument may be a root canal instrument as described herein, wherein the treatment with the dental instrument in step a) is provided by treating a root canal of the healthy tooth with said root canal instrument, thereby yielding a treated root canal.

The dental instrument in the method described herein may be operated at various rotation frequencies. For instance, the dental instrument may be operated at a rotation frequency from 200 to 2000 rpm such as from 200 to 1500 rpm, from 200 to 500 rpm or from 500 to 1500 rpm. In this document rpm stands for revolutions per minute.

It will be appreciated that the rotation frequency of the dental instrument may affect the creation of a smear layer. Thus, when operated at certain frequencies the dental instrument may provide dentin tissue lacking a smear layer while use of the same dental instrument at other frequencies may provide dentin tissue with a smear layer. When testing the dentin tissue in the method described herein it is therefore advisable to make a note about the rotation frequency being used.

In a further aspect there is provided a kit of parts for detection and/or removal of carious dentin tissue. The kit of parts comprises a composition comprising one or more hydrazine derivatives being a compound of formula (I)

RNHNH₂ (I).

R is a chemical group containing a chromophore or forming a chromophore with NHNH₂.

The kit of parts further comprises a dental instrument that provides a treated tooth free from staining in the method for testing for testing a dental instrument as described herein.

The dental instrument may be a dental drill that provides drilled dentin tissue free from staining in the method for testing a dental instrument as described herein.

The dental instrument may be a root canal instrument that provides treated dentin tissue free from staining in the method for testing a dental instrument as described herein.

The composition in (i) may be a preparation containing an active, caries-dissolving two-component liquid in the form of a first active, caries-dissolving component and the a second component which reduces the aggressiveness of the active component to mucous membranes, a gel substance and one or more hydrazine derivatives as described herein. The composition may be Carisolv®, Perioplus® or Perisolv including one or more hydrazine derivatives as described herein.

The method may further comprise one or more solutions for providing rinsing steps, wherein said one or more solutions may be water, brine or an aqueous pharmacologically acceptable solution.

The kit of parts may further comprise a flow apparatus for proving a fluid flow, such as an air flow, or a powder-like blasting to a tooth as described herein. The flow apparatus may also constitute said dental instrument as such.

The kit of parts may comprise instructions for detection and/or removal of carious dentin tissue.

In a further aspect there is provided a method for detection and removal of carious dentin tissue.

The method comprises the steps of:
if the carious dentin tissue comprises a smear layer(s), removing the smear layer(s) from the area of the carious dentin tissue
subjecting the carious dentin tissue of a tooth to treatment by a dental instrument, thereby yielding treated dentin tissue,
addition of a composition comprising one or more hydrazine derivative(s) to the dentin tissue,
examining if the treated dentin tissue free from a smear layer (s) is stained by the one or more hydrazine derivative(s), and
determining whether or not the treated tissue free from the smear layer (s) contains a carious dentin tissue, wherein stained dentin tissue by said one or more hydrazine derivative(s) indicates the presence of carious dentin tissue, and wherein a lack of stained dentin tissue by said one or more hydrazine derivative(s) indicates the absence of carious dentin tissue.

Said one or more hydrazine derivatives is a compound of formula (I)

RNHNH₂ (I)

wherein R is a chemical group containing a chromophore or forming a chromophore with NHNH₂.

Said one or more hydrazine derivatives may be any one of derivatives as will be further described in the detailed description or as defined in the claims.

The dental instrument may provide a treated dentin tissue free from staining in the method for testing a dental instrument as described herein.

The dental instrument may be a dental drill, and wherein the treatment with the dental instrument is provided by drilling with the dental drill, thereby yielding drilled dentin tissue.

The dental drill may be a drill as will be further described in the detailed description or as defined in the claims.

The dental instrument may be a root canal instrument, and wherein the treatment with the dental instrument provided by treating a root canal of the healthy tooth with said root canal instrument, thereby yielding a treated root canal.

The treatment by the dental instrument may provide for the removal of the smear layer that may be present in the carious dentin tissue.

The method may further comprise adding a preparation as described herein that contains an active, caries-dissolving two-component liquid in the form of a first active, caries-dissolving component and a second component which reduces the aggressiveness of the active component to mucous membranes, and a gel substance for use in removal of smear layer(s) from tooth enamel or dentin tissue.

The method may further comprise one or more rinsing steps, wherein rinsing is performed with water, brine or an aqueous pharmacologically acceptable solution.

The preparation may be removed by a fluid flow, such as an air flow, and/or by a powder-like blasting as described herein, wherein carious dentin is removed. This may be provided with a flow apparatus as described herein. Such flow apparatus may constitute the dental instruments as such.

The preparation described herein may include said one or more hydrazine derivatives.

In a further aspect, there is provided a preparation containing an active, caries-dissolving two-component liquid in the form of a first active, caries-dissolving component and the a second component which reduces the aggressiveness of the active component to mucous membranes, and a gel substance for use in removal of smear layer(s) from healthy or carious dentin tissue. The preparation may an aqueous composition comprising a first active component NaOCl in a concentration of 1-2% (w/w), a second component having a pH between 9.5 and 10.5 and comprising a mixture of glutamic acid, leucine and lysine in a concentration of 0.5-1.5% (w/w), NaCl in a concentration of 0.5% (w/w), and high viscosity carboxymethyl cellulose gel in a concentration of 2.5-5% (w/w). The preparations may be Carisolv®, Perioplus® or Perisolv as described herein below.

In still a further aspect there is provided a composition comprising one or more hydrazine derivatives for use in the treatment of removal of carious dentin tissue free from smear layer, wherein the treatment comprises the steps of: (i) addition of the composition comprising one or more hydrazine derivatives to carious dentin tissue free from smear layer, and (ii) subjecting the carious dentin tissue free from smear layer to drilling by a dental drill that provides drilled dentin tissue free from staining in the method according to the first aspect.

In a further aspect there is provided a composition comprising one or more hydrazine derivative(s) for use in the detection of smear layer(s) in healthy dentin tissue.

In a further aspect there is provided a composition comprising one or more hydrazine derivative(s) for use in the detection of carious dentin tissue free from smear layer(s).

In a further aspect there is provided a composition comprising one or more hydrazine derivative(s) for the manufacture of a detection agent for detection of smear layer(s) in healthy dentin tissue.

In a further aspect there is provided a composition comprising one or more hydrazine derivative(s) for the manufacture of a detection agent for detection of carious dentin tissue free from smear layer(s).

In a further aspect there is provided a method of detecting smear layer(s) in healthy dentin tissue, said method comprising the steps of:

(i) addition of a composition comprising one or more hydrazine derivative(s) onto the dentin tissue and
(ii) visual examination of the dentin tissue stained by the composition comprising the one or more hydrazine derivative(s) in step (b). The dentin tissue under examination may be treated with acid etching as known in the art or Carisolv® prior to step (i).

In a further aspect there is provided a method for detection of carious dentin tissue free of smear layer comprising the steps of:

(i) addition of a composition comprising one or more hydrazine derivative(s) onto the carious dentin tissue free of smear layer, and
(ii) visual examination of the dentin tissue stained by the composition comprising the one or more hydrazine derivative(s) in step (b).

In a further aspect there is provided a method for removal of carious dentin tissue free from smear layer(s) comprising the steps of:

(i) addition of a composition comprising one or more hydrazine derivative(s) to the carious dentin tissue free from smear layer(s), and
(ii) subjecting the carious dentin tissue free from smear layer to treatment by a dental instrument as described herein that provides treated dentin tissue free from staining in the method according to the first aspect.

### BRIEF DESCRIPTION OF THE FIGURES

FIG. 1a shows deconvoluted FTIR spectra for healthy dentin tissue (curve A and curve B) collected from two teeth and carious dentin tissue (curve C and curve D) collected from the same two teeth

FIG. 1b shows FTIR spectra where curve B (healthy dentin tissue) has been subtracted from curve C and curve D in FIG. 1a resulting in curve C and curve D″.

FIG. 2a shows staining of a tooth slice treated with 5-(2-2-hydrazinyl-2-oxoethylthio)acetamido)-2-(3-hydroxy-6-oxo-6H-xanthen-9-yl)benzoic acid after incubation with NaCl and NaOH.

FIG. 2b shows staining of a tooth slice treated with Lucifer Yellow sodium salt after treatment with NaCl and NaOH. The stained area is indicated with a circle and (1).

FIG. 2c shows staining of a tooth slice treated with Alexa Fluor 594® after treatment with NaCl and NaOH.

FIG. 3a shows the resulting FTIR spectra between 3700 and 2600 cm⁻¹ for healthy dentin tissue samples S1 (reference sample), S2 and S3. S2 and S3 were treated with Lucifer Yellow.

FIG. 3b shows the resulting FTIR spectra between 3700 and 2600 cm⁻¹ for carious dentin tissue samples S4 (reference sample), S5 and S6. S5 and S6 were treated with Lucifer Yellow.

FIG. 3c shows the resulting FTIR spectra between 1800 and 400 cm⁻¹ for healthy dentin tissue samples S1 (reference sample), S2 and S3. S2 and S3 were treated with Lucifer Yellow.

FIG. 3d shows the resulting FTIR spectra between 1800 and 400 cm⁻¹ for carious dentin tissue samples S4 (reference sample), S5 and S6. S5 and 86 were treated with Lucifer Yellow.

FIG. 3e shows the resulting FTIR spectra between 1800-1680 cm⁻¹ for carious dentin tissue samples S4 (ref), S5 and S6. S5 and S6 were treated with Lucifer Yellow and compared with healthy dentin tissue S1 (reference sample).

FIG. 4a shows positive TOF-SIMS spectra for carious dentin tissue. The upper spectrum shows carious dentin tissue. The lower spectrum shows carious dentin tissue treated with the hydrazine derivative Lucifer Yellow.

FIG. 4b shows positive TOF-SIMS spectra for healthy dentin tissue. The upper spectrum shows healthy dentin tissue. The lower spectrum shows healthy dentin tissue treated with the hydrazine derivative Lucifer Yellow.

FIG. 5 shows a schematic side view of a dental drill having a substantially spherical head.

FIG. 6 shows a side view, analogue to FIG. 5, with a substantially pear-shaped head.

FIGS. 7 and 8 show front side plan views onto two different embodiments.

FIGS. 9 and 10 show sectional views in a radial plane of the embodiments shown in FIGS. 7 and 8.

FIGS. 11 and 12 show schematic side views of further embodiments, wherein the cutting edges are provides with at least one cross-cut.

FIG. 13 shows a perspective side view of an embodiment, analogue to FIG. 1, wherein each cutting edge comprises a cross-cut.

FIGS. 14 and 15 show detailed views of an embodiment in which each cutting edge comprises a plurality of cross-cuts.

### DETAILED DESCRIPTION

A smear layer is a layer with small crystalline characteristics comprising organic particle debris. It appears on the surface of teeth that have undergone dental instrumentation procedures, including root planing and cutting done with a dental bur.

By selective removal of carious dentin tissue is meant that only carious dentin tissue is removed; i.e. nothing or virtually nothing else is removed. Accordingly, no or very little healthy dentin tissue is removed.

By selective labelling or detection is meant that the labeling or detection takes place only between the carious dentin tissue and the hydrazine derivative without labeling or detection of the healthy dentin tissue. Since addition of a hydrazine derivative to carious dentin tissue leads to both labelling and detection the terms label/labelling and detection/detecting will be used interchangeably in this document.

By irreversible labeling or detection is meant that a hydrazine derivative that has reacted with carious dentin tissue will not be removed during rinsing with for instance water, an aqueous solution of NaCl or an aqueous solution of NaOH.

While not wishing to be bound by any specific theory, it is believed that the irreversible labelling of the carious dentin tissue with a hydrazine derivative may be due to the formation of one or more covalent bonds between the carious tissue and the hydrazine derivative.

Since carious dentin tissue contains ester groups and healthy dentin tissue contains amide groups hydrazine derivatives would have been expected to react with the carbonyl groups in both of these, albeit more slowly with the amides. Compared to tooth enamel, healthy dentin tissue is softer and more permeable. Healthy dentin tissue would be expected to receive and retain dye comprising hydrazine derivatives due to its permeable nature. The permeable nature of healthy dentin is due to its composition as well as the presence of dentin tubules. The large amount of collagen in dentin tissue makes it much softer than tooth enamel and therefore likely to be prone to staining. The dentin tubules may transport fluids such as liquid dyes. Therefore, the permeable nature of the largely collagen containing healthy dentin tissue surrounding the carious dentin tissue would make staining likely to take place both in healthy dentin and carious dentin, i.e. there would be no selective staining of the carious dentin tissue. However, the inventors of the present invention observed no reaction whatsoever when a hydrazine derivative was mixed with healthy dentin tissue.

The irreversible labelling of the carious dentin tissue with the hydrazine derivative, possibly through the formation of covalent bonds between carious dentin tissue and the hydrazine derivative, has the advantage that the excess of hydrazine derivative may easily be removed by rinsing without affecting the extent to which the carious dentin tissue is labelled. This allows for more precise caries removal, i.e. removal of as much carious dentin tissue as possible without affecting healthy dentin tissue, since substantially all of the carious dentin tissue will remain labelled after rinsing.

In contrast, a rinsing step in conventional methods using dyes forming non-covalent bonds with carious dentin tissue may easily lead to removal of substantial amounts of dye in addition to the removal of excess of dye resulting in labelling of only a part of the carious dentin tissue. It has been found by the inventors of the present invention that rinsing carious dentin tissue labelled with a dye such as Acid Red forming only electrostatic bonds to the carious dentin tissue results not only in removal of excess dye but also in release of the dye bonded to the carious dentin tissue; i.e. after rinsing only part of the carious dentin tissue is labelled with the dye. Of course, such a partial labelling is not helpful when it is desired to remove all carious dentin tissue.

The fact that the one or more hydrazine derivative(s) label(s) carious dentin tissue in a selective and reversible way make it/them useful for detecting the presence or absence of carious dentin tissue. Thus, there is a two-fold purpose of using hydrazine derivative(s).

Firstly, the hydrazine derivative(s) may be used for labelling carious dentin tissue thereby facilitating for the skilled practitioner to see the diseased, i.e. carious, tissue requiring removal.

Secondly, the hydrazine derivatives may be used for detecting the presence or absence of carious dentin tissue subsequent to, for instance, measures taken by a dentist to remove carious dentin tissue. Therefore, when carious dentin tissue is removed using a dental instrument such as a dental drill it is necessary to ensure that the dental instrument does not generate a smear layer.

In this document, the terms labelling and detection are used interchangeably. In both cases, however, it is essential that the carious dentin tissue is free from smear layer(s) since the inventors of the present invention have found that hydrazine derivative(s) stain(s) smear layers.

Accordingly, in a first aspect there is provided a method for testing a dental instrument. The method comprises the steps of:
a) subjecting healthy dentin tissue free from smear layer(s) of an extracted healthy tooth to treatment with a dental instrument, thereby yielding treated dentin tissue,
b) addition of a composition comprising one or more hydrazine derivative(s) to the treated dentin tissue obtained in step a), and
c) examining if the treated dentin tissue in step b) is stained by the one or more hydrazine derivative(s), and
d) determining whether or not the treated dentin tissue contains a smear layer, wherein a stained dentin tissue in step c) indicates the presence of a smear layer, and wherein a lack of stained dentin tissue in step (c) and tissue therein indicates the absence of a smear layer.

Said one or more hydrazine derivatives is a compound of formula (I)

RNHNH₂ (I)

wherein R is a chemical group containing a chromophore or forming a chromophore with NHNH₂.

The dental instrument may be a dental drill. Examples of such drills will be described in more detail hereinbelow.

The dental instrument may be a root canal instrument. An example of such a root canal instrument is a file as the one sold by Komet Dental Gebr. Brasseler GmbH & Co.KG.

The dental instrument in the method described herein may be operated at various rotation frequencies. For instance, the dental instrument may be operated at a rotation frequency from 200 to 2000 rpm such as from 200 to 1500 rpm, from 200 to 500 rpm or from 500 to 1500 rpm. In this document rpm stands for revolutions per minute.

It will be appreciated that the rotation frequency of the dental instrument may affect the creation of a smear layer. Thus, when operated at certain frequencies the dental instrument may provide dentin tissue lacking a smear layer while use of the same dental instrument at other frequencies may provide dentin tissue with a smear layer. When testing the dentin tissue in the method described herein it is therefore advisable to make a note about the rotation frequency being used.

The dental instrument will in most cases be described herein by the use of a dental drill. The skilled person will appreciate that the dental drill in general may be interchanged with any other dental instrument, such as a root canal instrument.

The extracted healthy tooth may have no or little previous dental restoration. Staining of the drilled dentin tissue implies that the dental drill under examination yields smear layer(s). No staining of the drilled dentin tissue implies that the dental drill under examination yields no smear layer(s).

It will be appreciated that the method for testing a drill (dental instrument) as described herein may comprise one or more rinsing steps. Rinsing may take place with water, brine or an aqueous physiologically acceptable solution. Further, the examination in step (c) may take place using an instrument. The instrument may operate between 10 and 400 nm, 200 and 400 nm, 280 and 400 nm, 200 and 500 nm, 450 and 500 nm or 460 and 470 nm. An example of such an instrument is PoliLed SN06LP1121, Faro.

In an embodiment of the invention the method for testing a dental drill as described hereinbefore or hereinafter is a method for testing the formation of smear layer(s) by a dental instrument as here exemplified by a dental drill.

As shown in Example 8 herein dentin tissue free from smear layer(s) may be provided by treatment of dentin tissue comprising smear layer(s) with the preparation Carisolv®.

Accordingly, in a further aspect there is provided a preparation containing an active, caries-dissolving two-component liquid in the form of a first active, caries-dissolving component and the a second component which reduces the aggressiveness of the active component to mucous membranes, and a gel substance for use in removal of smear layer(s) from tooth enamel or dentin tissue. The dentin tissue may be healthy or carious dentin tissue. The first active, caries-dissolving component may be NaOCl, KOCl or Ca(ClO)₂. The preparation may be Carisolv®, PerioPlus® or Perisolv.

In still a further aspect, there is provided a preparation containing an active, caries-dissolving two-component liquid in the form of a first active, caries-dissolving component and the a second component which reduces the aggressiveness of the active component to mucous membranes, and a gel substance for use in the manufacture of a removal agent for removal of smear layer(s) from tooth enamel or dentin tissue. The dentin tissue may be healthy or carious dentin tissue. The first active, caries-dissolving component may be NaOCl, KOCl or Ca(ClO)₂. The preparation may be Carisolv®, PerioPlus® or Perisolv.

The method as defined herein wherein the healthy dentin tissue free from smear layer(s) may be obtained by subjecting healthy dentin tissue to acid etching or to treatment with a preparation containing an active, caries-dissolving two-component liquid in the form of a first active, caries-dissolving component and the a second component which reduces the aggressiveness of the active component to mucous membranes, and a gel substance. The preparation may be Carisolv® or PerioPlus®.

The preparation Carisolv® is an aqueous composition comprising a first active component NaOCl in a concentration of 1-2% (w/w), a second component having a pH between 9.5 and 10.5 and comprising a mixture of glutamic acid, leucine and lysine in a concentration of 0.5-1.5% (w/w), NaCl in a concentration of 0.5% (w/w), and high viscosity carboxymethyl cellulose gel in a concentration of 2.5-5% (w/w). The composition may further comprise Na₂-Erythrocine (coloring agent). High viscosity of carboxymethyl cellulose is defined as 1500-3000 centi poise (cP) in 1% H₂O (25° C.).

PerioPlus® or Perisolv is an aqueous composition comprising a first active component NaOCl in a concentration of 1-2% (w/w), a second component having a pH between 9.5 and 10.5 and comprising a mixture of glutamic acid, leucine and lysine in a concentration of 0.5-1.5% (w/w), NaCl in a concentration of 0.5% (w/w), TiO₂ in a concentration of 0.03% (w/w) and medium viscosity carboxymethyl cellulose gel in a concentration of 2.5-5% (w/w). Medium viscosity of carboxymethyl cellulose gel is defined as 400-800 centi poise (cP) in 2% H₂O (25° C.). The composition may further comprise Na₂-Erythrocine (coloring agent).

The method may further comprise one or more rinsing steps, wherein rinsing is performed with water, brine or an aqueous pharmacologically acceptable solution.

The preparation or compositions that have been applied to a tooth or teeth as described herein may be removed from the tooth or teeth by a fluid flow, such as an air flow, and/or by a powder-like blasting. In this way a healthy tooth with dentin tissue free from smear layer(s) and/or removal of carious dentin tissue may be obtained. The application of a fluid flow and/or sand-like blasting may be provided by a flow apparatus as is known in the art and that skilled person finds suitable for providing said flow or blasting. An example of a suitable instrument is the AIR FLOW® instrument that is available from Electro Medical Systems (EMS, e.g. EMS SA). The flow apparatus may constitute the dental instruments as such.

Said one or more hydrazine derivatives is a compound of formula (I)

RNHNH₂ (I)

wherein R is a chemical group containing a chromophore or forming a chromophore with NHNH₂. The chromophore may be formed before, upon or after reaction of the compound of formula (I) with carious dentin tissue.

In this document, the term “chromophore” means the part of the molecule resulting in its colour. The colour arises when that part of the molecule absorbs certain wavelengths of visible light and transmits or reflects others.

The hydrazine derivative of formula (I) is, either by itself or after reaction with carious dentin tissue, visible to the naked human eye in daylight, i.e. the hydrazine derivative reflects visible light. In this document, visible light is defined as electromagnetic radiation having a wavelength in the range of about 380 nm to about 750 nm. If desired, an instrument may be used in order to enhance visibility. The instrument may operate between 10 and 400 nm, 200 and 400 nm, 280 and 400 nm., 200 and 500 nm, 450 and 500 nm or 460 and 470 nm. An example of such an instrument is PoliLed SN06LP1121, Faro.

It is to be understood that the hydrazine derivative of formula (I) may be, for instance, a carbazide or a hydrazide. The hydrazine derivative according to formula (I) or as further specified herein may be the derivative(s) used in any aspect of the invention.

There is provided a method as defined herein wherein the one or more compounds being a hydrazine derivative is a compound of formula (Ia):

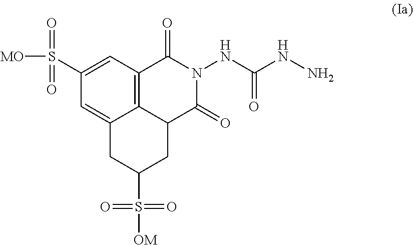

wherein M represents a monovalent metal ion selected from Li⁺, K⁺ and Na⁺. When M is K⁺ the compound of formula (Ia) is denominated Lucifer Yellow.

There is also provided a method as defined herein wherein the one or more compounds being a hydrazine derivative is a compound of formula (Ib):

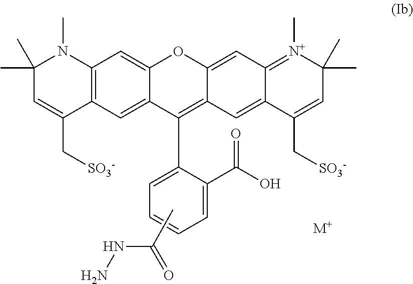

wherein M⁺ represents a monovalent metal ion selected from Li⁺, K⁺ and Na⁺.

The trade name of the compound of formula (Ib) when M⁺ is Na⁺ is Alexa Fluor® 594 hydrazide sodium salt.

The chemical name for the compound of formula (Ib) is 6-(2-carboxy-5-(hydrazinecarbonyl)phenyl)-1,2,2,10,10,11-hexamethyl-2,10-dihydro-1H-pyrano[3,2-g]diquinoline-11-ium-4,8-diyl)methanesulfonate.

There is also provided a method as defined herein wherein the one or more compounds being a hydrazine derivative is a compound of formula (Ic):

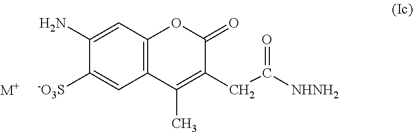

wherein M⁺ represents a monovalent metal ion selected from Li⁺, K⁺ and Na⁺

The trade name of the compound of formula (Ic) when M⁺ is Na⁺ is Alexa 350.

There is also provided a method as defined herein wherein the one or more compounds being a hydrazine derivative is a compound of formula (Id):

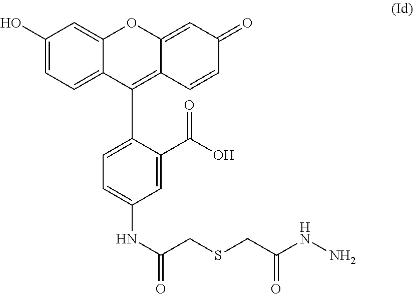 [SMILES: NNC(=O)CSCC(=O)Nc1ccc(-c2c3ccc(=O)cc-3oc3cc(O)ccc23)c(C(=O)O)c1]

The trade name of the compound of formula (Id) is 5-(((2-(carbohydrazino)methyl)thio)acetyl)aminofluorescein, whereas the chemical name suggested by Chemdraw (CS Chemdraw Ultra, Cambridge Soft, USA) for the compound (Id) is 5-(2-2-hydrazinyl-2-oxoethylthio)acetamido)-2-(3-hydroxy-6-oxo-6H-xanthen-9-yl)benzoic acid.

Further examples of hydrazine derivatives are HiLyte Fluor™ 488 hydrazide, HiLyte Fluor™ 555 hydrazide, HiLyte Fluor™ 594 hydrazide, HiLyte Fluor™ 647 hydrazide, HiLyte Fluor™ 680 hydrazide.

There is also provided a method for testing a dental instrument such as a dental drill, the method comprising the steps of:

(a) subjecting healthy dentin tissue free from smear layer(s) of an extracted healthy tooth to treatment with the dental instrument, e.g. drilling with the dental drill, thereby yielding treated dentin tissue,
(b) addition of a composition comprising a hydrazine derivative according to formula (Ia) to the treated dentin tissue obtained in step (a), and
(c) examining if the treated dentin tissue in step (b) is stained by the one or more hydrazine derivative(s).

There is also provided a method for testing the formation of smear layer(s) by a dental instrument such as a dental drill comprising the steps of:

(a) subjecting healthy dentin tissue free from smear layer(s) of an extracted healthy tooth to treatment with the dental instrument, e.g. drilling with the dental drill, thereby yielding a treated dentin tissue,
(b) addition of a composition comprising a hydrazine derivative according to formula (Ia) to the treated dentin tissue obtained in step (a), and
(c) examining if the treated dentin tissue in step (b) is stained by the one or more hydrazine derivative(s).

There is also provided a kit of parts for detection and/or removal of carious dentin tissue comprising: (i) a composition comprising one or more hydrazine derivatives, and (ii) a dental instrument such as a dental drill that provides a treated dentin tissue free from staining in the method as described herein.

The composition mentioned herein may comprise a solvent such as water, glycerine, propylene glycol, mineral oil, ethanol, acetone, polysorbate, 80, or any like solvent.

The composition may further comprise an anesthetic such as lidocaine or benzocaine.

The kit of parts mentioned herein may also comprise a tool for applying the composition comprising the one or more hydrazine derivative(s). Examples of such tools include, but are not limited to, a brush, a syringe, a pen, a fibrous pellet or any fibrous web material.

The kit of parts mentioned herein may contain or be used together with a rinsing solution. For instance, the rinsing solution may be water, brine or any physiologically aqueous solution.

The kit of parts mentioned herein may also comprise a preparation containing an active, caries-dissolving two-component liquid in the form of a first active, caries-dissolving component and a second component which reduces the aggressiveness of the active component to mucous membranes, and a gel substance for use in removal of smear layer(s) from healthy or carious dentin tissue. The preparation may be mixed with the one or more hydrazine derivatives described herein. The preparation may be Carisolv®, PerioPlus® or Perisolv.

Thus, there is also provided a kit of parts for detection and/or removal of carious dentin tissue comprising: (i) a preparation containing an active, caries-dissolving two-component liquid in the form of a first active, caries-dissolving component and the a second component which reduces the aggressiveness of the active component to mucous membranes, a gel substance and one or more hydrazine derivatives as described herein, and (ii) a dental drill that provides drilled dentin tissue free from staining in the method as described herein. The preparation may be Carisolv®, PerioPlus® or Perisolv including one or more hydrazine derivatives described herein. The one or more hydrazine derivatives may be a compound of formula (I) such as a compound (Ia), (Ib), (Ic) and/or (Id).

The dental drills described herein are particularly suited for removing diseased dentin, i.e. carious dentin tissue, from cavities in case of a cavity preparation, wherein diseased dentin is removed, however, sane dentin (i.e. healthy dentin tissue) is preserved.

There is also provided a kit of parts as described herein wherein the one or more hydrazine derivatives is/are as described hereinbefore or hereinbelow.

There is provided a kit of parts as described herein wherein the dental drill comprises a head having a rounded basic shape, and at least two cutting edges at a front side that merge into a transitional cutting edge, said dental drill comprising plastic having a hardness between 60 and 250 Knoop. The dental drill may be made of a plastic comprising one or more of the following: polyoximethylene (POM), polycarbonate (PC), polyetherimide (PE), polyethersulfone (PES), polysulfone (PSU), polyamiddiimide (PAI), polyfenilensulfone (PPSU), and polyetheretherketone (PEEK). The plastic may be reinforced with fibers.

There is also provided a kit of parts as described herein comprising: (a) a composition comprising a compound of formula (Ia), and (b) a dental drill comprising a head having a rounded basic shape, and at least two cutting edges at a front side that merge into a transitional cutting edge, said dental drill comprising plastic having a hardness between 60 and 250 Knoop.

In this document, the unit Knoop is mentioned in the context of hardness of a material such as a plastic. The inventive measure of hardness according to Knoop refers to DIN EN ISO 4545. Similar to the Vickers examination, the hardness examination according to Knoop uses a diamond peak having a rhombic shape, which has an acute angle of 172.5° for the long side and 130° for the short side.

There is also provided a kit of parts as described herein wherein the dental drill the dental drill comprises a head having a plurality of cutting edges, the head further having an at least rounded basic shape, wherein two of the cutting edges merge into a transitional cutting edge at a front side, and wherein the dental drill is made of a ceramic material and a cutting edge chipping is between about 2.5 and 7 μm and a cutting edge base has a radius between about 0.03 and 0.12 mm. In this document, cutting edge chipping means the depth of the notch resulting during the manufacturing of the dental drill and the generation of the cutting edge. The ceramic material may comprise zirconium oxide or zirconium oxide with additives.

There is also provided a kit of parts as described herein comprising: (a) a composition comprising a compound of formula (Ia), and (b) a dental drill comprising a head having a plurality of cutting edges, the head further having an at least rounded basic shape, wherein two of the cutting edges merge into a transitional cutting edge at a front side, and wherein the dental drill is made of a ceramic material and a cutting edge chipping is between about 2.5 and 7 μm and a cutting edge base has a radius between about 0.03 and 0.12 mm.

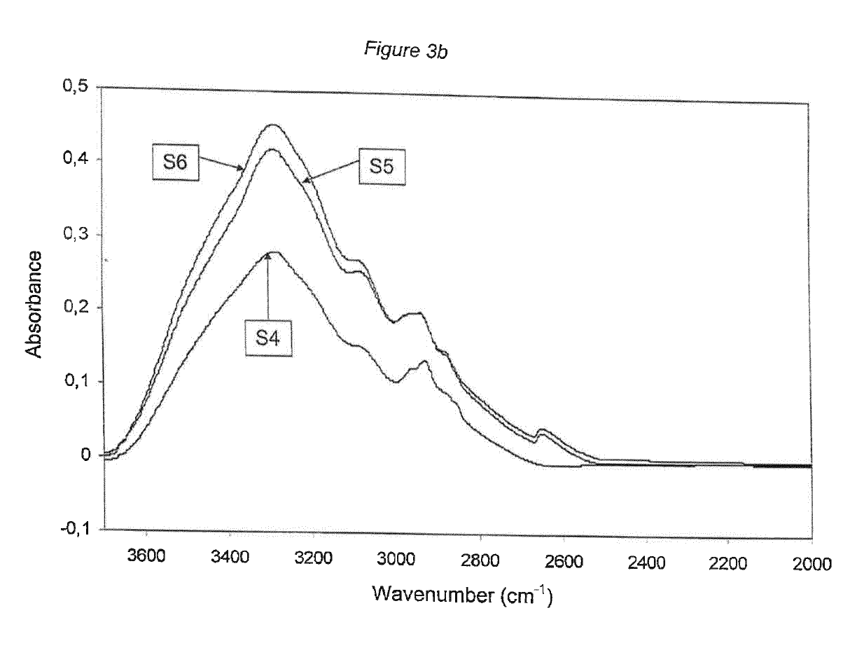

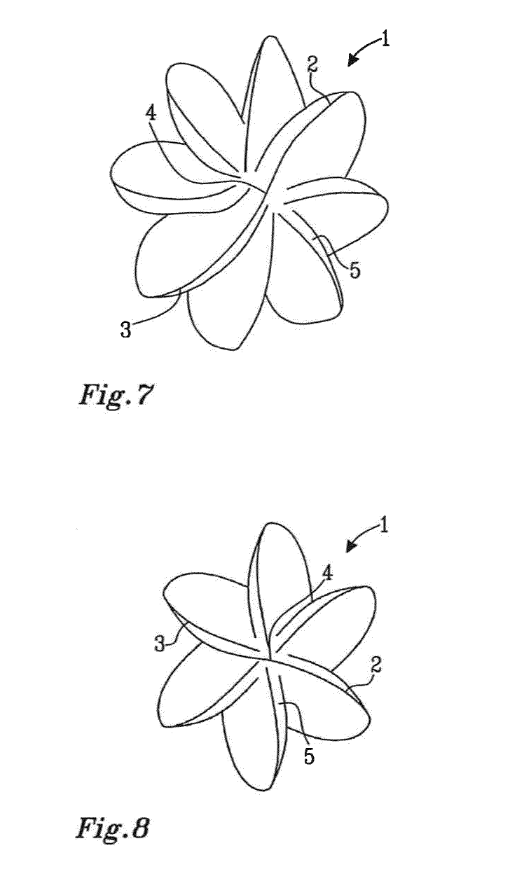

The dental drills as described herein are illustrated in FIGS. 5-15.

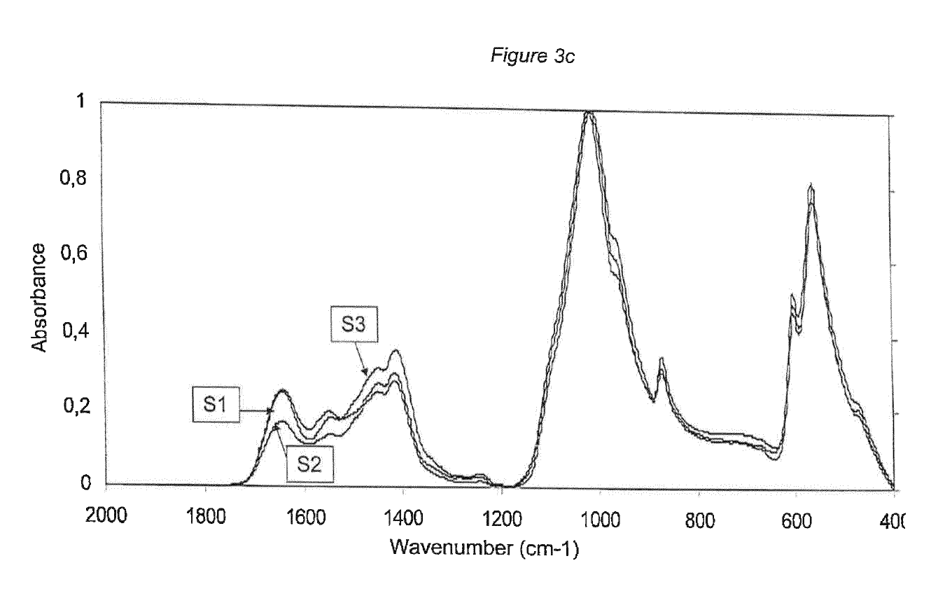

As results from FIGS. 5 and 6, the dental drill comprises a head 1 attached to a shaft 6 and integrally connected therewith. The number of cutting edges as well as the shape of the head depends on the respective purpose of use and the respective physical form.

FIG. 5 shows a substantially spherical head, whereas the head according to FIG. 6 is substantially pear-shaped.

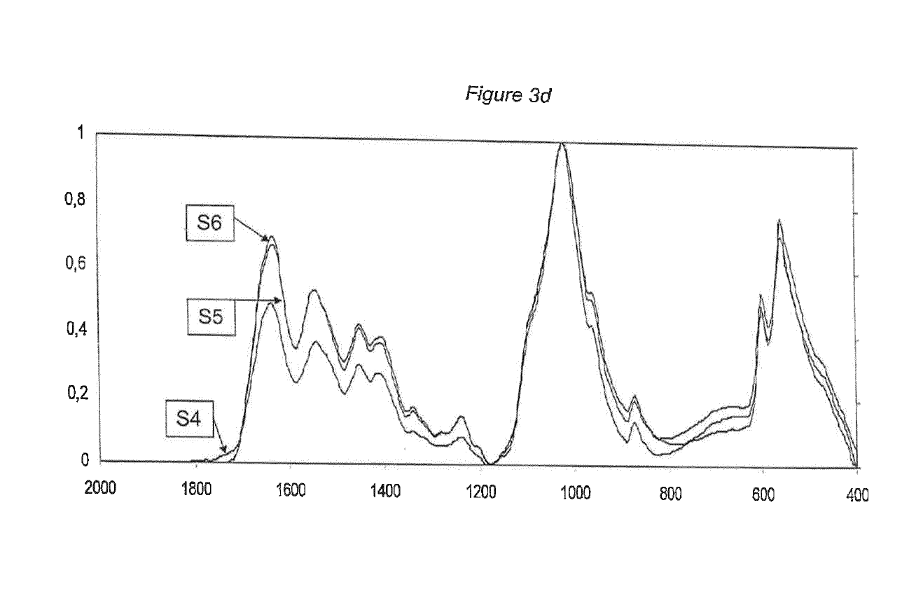

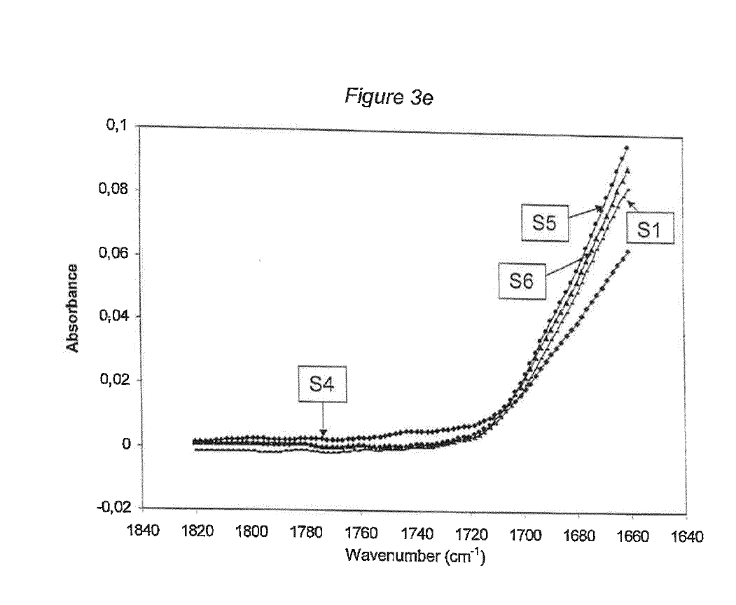

FIGS. 7 and 8 respectively show front views of two different embodiments. The embodiment of FIG. 7 comprises in total eight cutting edges. Thereof, two cutting edges 2, 3 are formed such that they merge into each other at the front sides and form a transitional cutting edge or cross cutting edge 4.

In the embodiment according to FIG. 8, six cutting edges are provided in total, wherein the cutting edges designated with reference numerals 2 and 3 merge into a transitional cutting edge or cross cutting edge 4.

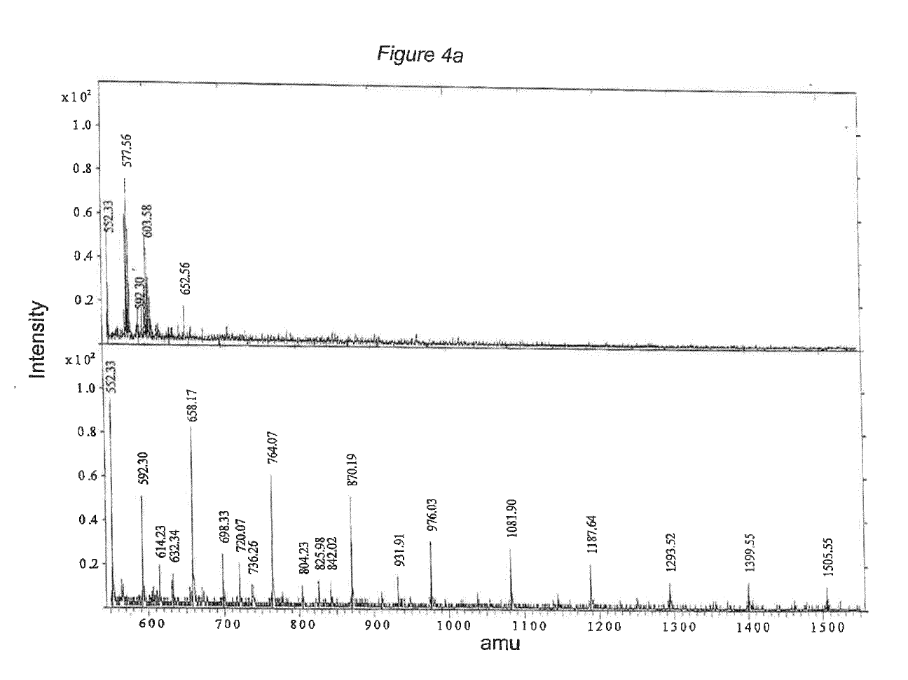

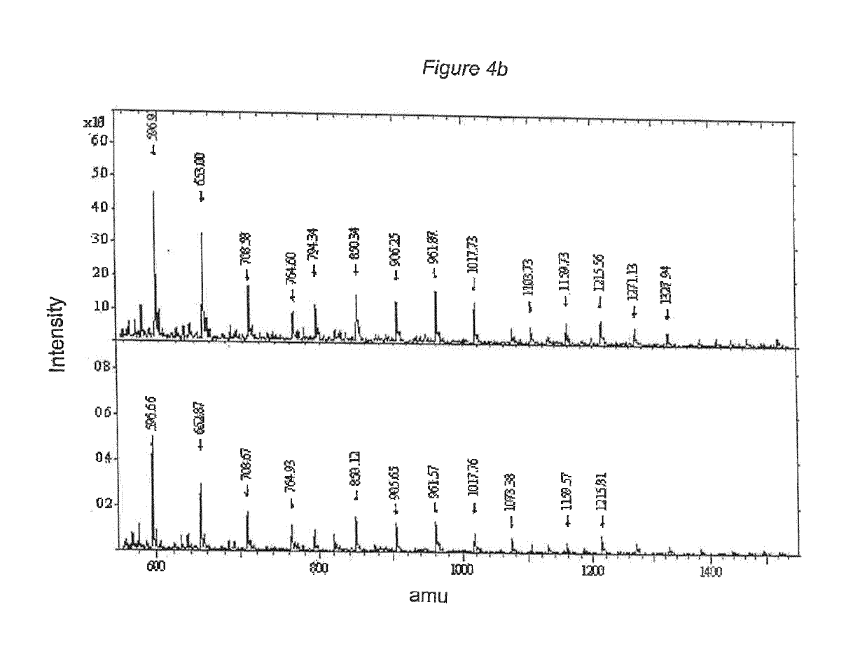

FIGS. 9 and 10 respectively show sectional views in the radial plane (with respect to the rotary axis of the dental drill). FIG. 9 corresponds to the embodiment of FIG. 8, whereas FIG. 10 corresponds to the embodiment of FIG. 7. According to the invention, it is provided that the cutting edge base 5 has a radius between 0.03 and 0.12 mm. Therewith, a rounding of the transitional areas is given, which reduces notch tensions and prevents the deposition of chips during operation.

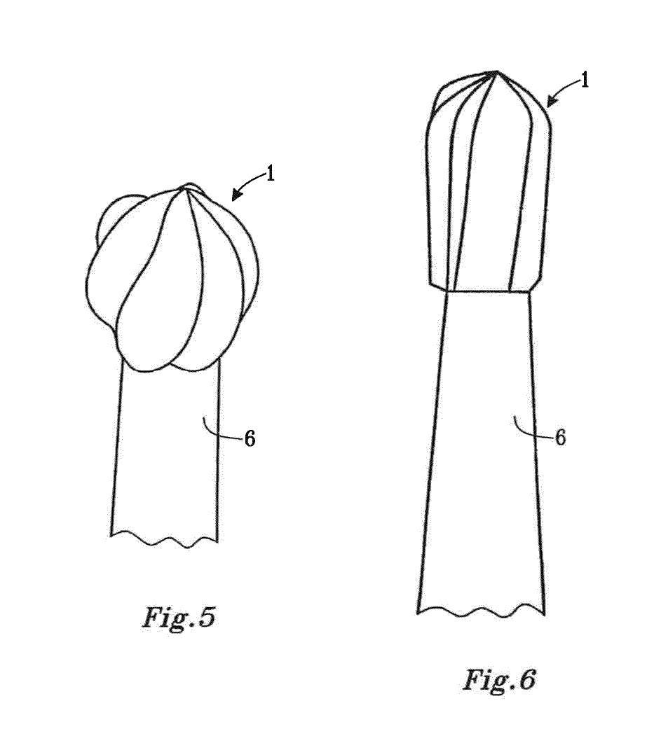

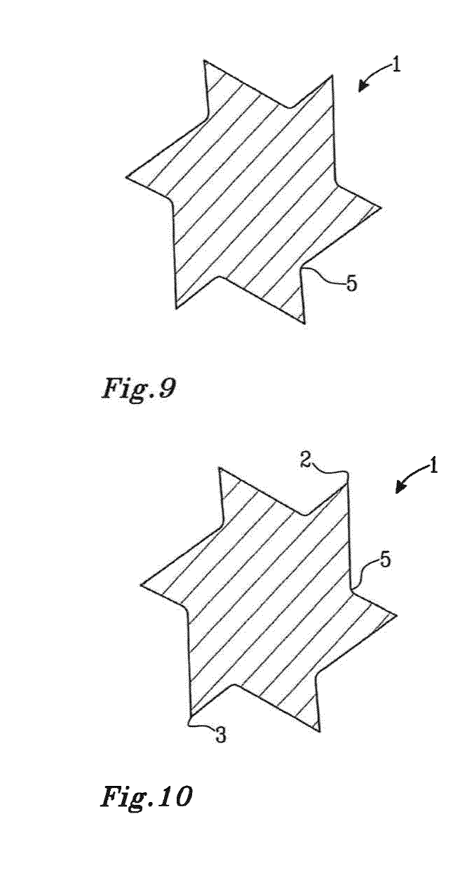

FIGS. 11 to 15 respectively show embodiments having a spherical head 1, wherein at least one cross-cut is provided in each of the cutting edges 2, 3. FIG. 13, in particular, shows an embodiment in which a cross-cut 7 having the shape of a depression-like rounded recess is provided at each of the cutting edges 2, 3.

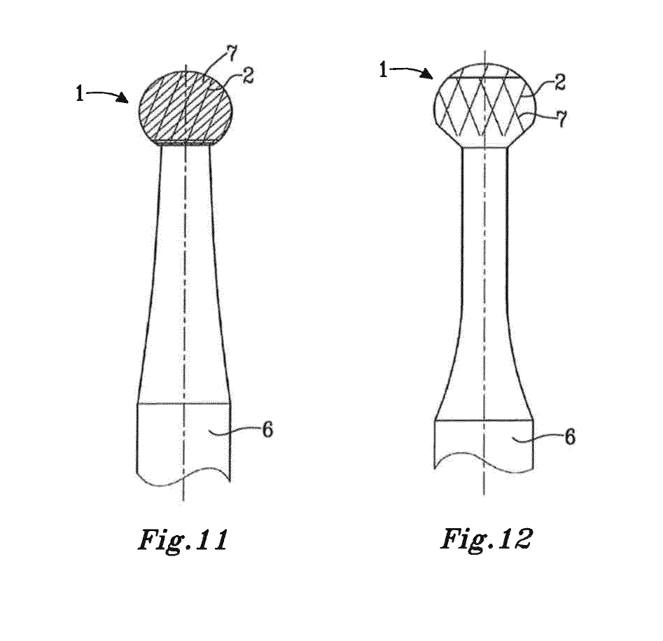

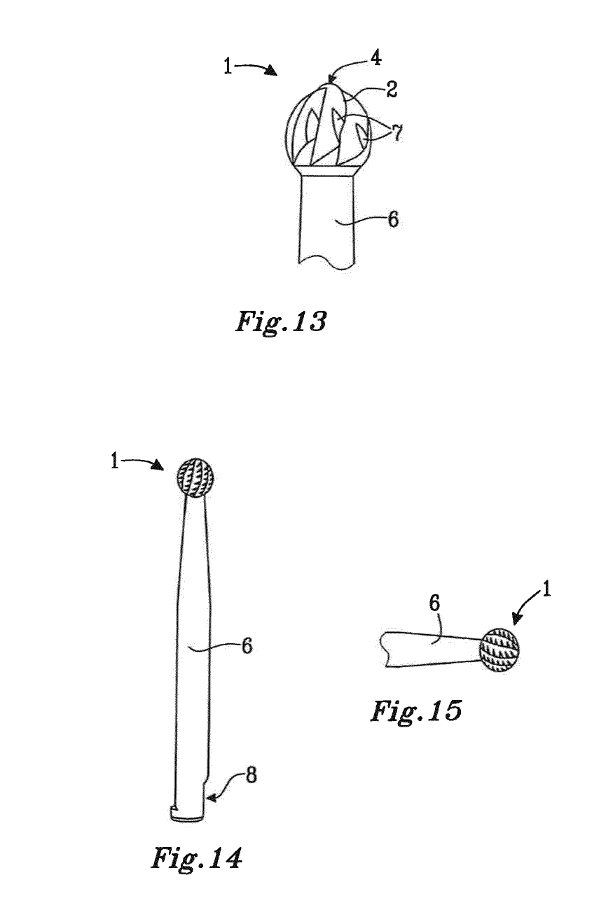

FIGS. 14 and 15 show an embodiment in which each cutting edge is provided with a plurality of cross-cuts. Same may be arranged to be helically circumferential around the longitudinal axis. The cross-cuts 7 may be arranged in a single row (as shown in FIG. 13) or a multi row (as shown in FIGS. 14 and 15).

FIG. 14 further shows that a chucking portion 8 for the dental drill is formed at the lower end of the shaft 6, which comprises an oblateness as well as a circumferential fixing groove, as it is pre-known from the state of the art.

### LIST OF REFERENCE NUMERALS IN FIGS. 5-15

1 head
2 cutting edge
3 cutting edge
4 transitional cutting edge/cross cutting edge
5 cutting edge base
6 shaft
7 cross-cut
8 chucking portion

The kit of parts as described herein may comprise: (a) a composition comprising a compound of formula (Ia), and (b) a dental instrument in the form of a root canal instrument as described herein.

The kit of parts may comprise a flow apparatus for providing a fluid flow, such as an air flow, or a sand-like blasting to a tooth so as to remove a preparation, smear layer and/or carious dentin tissue as described herein. The type of apparatus to use is known to the skilled person in view of intended use as described herein and the knowledge in the art.

The kit of parts may also comprise instructions for detection and/or removal of carious dentin tissue.

In a further aspect there is provided a method for detection and removal of carious dentin tissue, The method comprises the steps of:
if the carious dentin tissue comprises a smear layer(s), removing the smear layer(s) from the area of the carious dentin tissue
subjecting the carious dentin tissue of a tooth to treatment by an dental instrument, thereby yielding treated dentin tissue,
addition of a composition comprising one or more hydrazine derivative(s) to the dentin tissue,
examining if the treated dentin tissue free from a smear layer (s) is stained by the one or more hydrazine derivative(s), and
determining whether or not the treated tissue free from the smear layer (s) contains a carious dentin tissue, wherein stained dentin tissue by said one or more hydrazine derivative(s) indicates the presence of carious dentin tissue, and wherein a lack of stained dentin tissue by said one or more hydrazine derivative(s) indicates the absence of carious dentin tissue.

Said one or more hydrazine derivatives is a compound of formula (I)

RNHNH₂ (I)

wherein R is a chemical group containing a chromophore or forming a chromophore with NHHN2.

Said one or more hydrazine derivatives may be any one of derivatives as will be further described in the detailed description or as defined in the claims.

The dental instrument may provide treated dentin tissue free from staining in the method for testing a dental instrument as described herein.

The dental instrument may be a dental drill, and wherein the treatment with the dental instrument is provided by drilling with the dental drill, thereby yielding drilled dentin tissue.

The dental drill may be a drill as is be further described herein.

The dental instrument may be a root canal instrument as described hereinabove, and wherein the treatment with the dental instrument may be provided by treating a root canal of the healthy tooth with said root canal instrument, thereby yielding a treated root canal.

The treatment by the dental instrument may provide for the removal of a smear layer that may be present in the carious dentin tissue.

The method may further comprise adding a preparation as described hereinabove that contains an active, caries-dissolving two-component liquid in the form of a first active, caries-dissolving component and the a second component which reduces the aggressiveness of the active component to mucous membranes, and a gel substance for use in removal of smear layer(s) from tooth enamel or dentin tissue.

The method may further comprise one or more rinsing steps, wherein rinsing may be performed with water, brine or an aqueous pharmacologically acceptable solution.

The preparation may be removed by a fluid flow, such as an air flow, and/or by a sand-like blasting as described hereinabove, wherein carious dentin is removed.

The preparation may include said one or more hydrazine derivatives of formula (I) as described herein.

There is also provided a composition comprising one or more hydrazine derivatives for use in the removal of carious dentin tissue free from smear layer(s), wherein the removal comprises the steps of: (i) addition of the composition comprising one or more hydrazine derivatives to carious dentin tissue free from smear layer(s), and (ii) subjecting the carious dentin tissue free from smear layer to drilling by a dental drill that provides drilled dentin tissue free from staining in the method according to the first aspect. The composition may further comprise a dye such as patent blue.

There is also provided a composition comprising one or more hydrazine derivative(s) for the manufacture of a detection agent for treating removal of carious dentin tissue free from smear layer(s), wherein the treatment comprises the steps of: (i) addition of the composition comprising one or more hydrazine derivatives to carious dentin tissue free from smear layer, and (ii) subjecting the carious dentin tissue free from smear layer to drilling by a dental drill that provides drilled dentin tissue free from staining in the method according to the first aspect. The composition may further comprise a dye such as patent blue.

There is also provided a composition for use in the removal of carious dentin tissue free from smear layer(s) or for the manufacture of a detection agent for treating removal of carious dentin tissue free from smear layer(s) as defined hereinbefore or hereinbelow wherein step (i) takes place prior to step (ii).

There is also provided a composition for use in the removal of carious dentin tissue free from smear layer(s) or for the manufacture of a detection agent for treating removal of carious dentin tissue free from smear layer(s) as defined hereinbefore or hereinbelow wherein step (ii) takes place prior to step (i).

There is also provided a composition for use in the removal of carious dentin tissue free from smear layer(s) or for the manufacture of a detection agent for treating removal of carious dentin tissue free from smear layer(s) as defined hereinbefore or hereinbelow wherein step (ii) takes place prior to step (i) and the carious dentin tissue comprises smear layer(s).

There is also provided a composition for use in the removal of carious dentin tissue free from smear layer(s) or for the manufacture of a detection agent for treating removal of carious dentin tissue free from smear layer(s) as defined hereinbefore or hereinbelow wherein the one or more hydrazine derivatives is a compound of formula (I) as described hereinbefore or hereinbelow.

There is also provided a composition for use in the removal of carious dentin tissue free from smear layer(s) or for the manufacture of a detection agent for removal of carious dentin tissue free from smear layer(s) as defined hereinbefore or hereinbelow wherein the one or more hydrazine derivatives is a compound of formula (Ia), (Ib), (Ic) or (Id) as described hereinbefore or hereinbelow.

There is also provided a composition for use in the removal of carious dentin tissue free from smear layer(s) or for the manufacture of a detection agent for removal of carious dentin tissue free from smear layer(s) as defined hereinbefore or hereinbelow wherein the dental drill comprises a head having a rounded basic shape, and at least two cutting edges at a front side that merge into a transitional cutting edge, said dental drill comprising plastic having a hardness between 60 and 250 Knoop. The dental drill may be made of a plastic comprising one or more of the following: polyoximethylene (POM), polycarbonate (PC), polyetherimide (PE), polyethersulfone (PES), polysulfone (PSU), polyamiddiimide (PAI), polyfenilensulfone (PPSU), and polyetheretherketone (PEEK). The plastic may be reinforced with fibers. The composition may further comprise a dye such as patent blue.

There is also provided a composition for use in the removal of carious dentin tissue free from smear layer(s) or for the manufacture of a detection agent for treating removal of carious dentin tissue free from smear layer(s) as defined hereinbefore or hereinbelow wherein the one or more hydrazine derivative(s) is a compound of formula (Ia), said compound of formula (Ia) being mixed with patent blue, and wherein the dental drill comprises a head having a rounded basic shape, and at least two cutting edges at a front side that merge into a transitional cutting edge, said dental drill comprising plastic having a hardness between 60 and 250 Knoop. The dental drill may be made of a plastic comprising one or more of the following: polyoximethylene (POM), polycarbonate (PC), polyetherimide (PE), polyethersulfone (PES), polysulfone (PSU), polyamiddiimide (PAI), polyfenilensulfone (PPSU), and polyetheretherketone (PEEK). The plastic may be reinforced with fibers.

There is also provided a composition for use in the removal of carious dentin tissue free from smear layer(s) or for the manufacture of a detection agent for treating removal of carious dentin tissue free from smear layer(s) as defined hereinbefore or hereinbelow wherein the dental drill comprises a head having a plurality of cutting edges, the head further having an at least rounded basic shape, wherein two of the cutting edges merge into a transitional cutting edge at a front side, and wherein the dental drill is made of a ceramic material and a cutting edge chipping is between about 2.5 and 7 μm and a cutting edge base has a radius between about 0.03 and 0.12 mm. The ceramic material may comprise zirconium oxide or zirconium oxide with additives. The composition may further comprise a dye such as patent blue.

There is also provided a composition for use in the removal of carious dentin tissue free from smear layer(s) or for the manufacture of a detection agent for treating removal of carious dentin tissue free from smear layer(s) as defined hereinbefore or hereinbelow wherein the one or more hydrazine derivatives is a compound of formula (Ia), said compound of formula (Ia) being mixed with patent blue, and wherein the dental drill comprises a head having a plurality of cutting edges, the head further having an at least rounded basic shape, wherein two of the cutting edges merge into a transitional cutting edge at a front side, and wherein the dental drill is made of a ceramic material and a cutting edge chipping is between about 2.5 and 7 μm and a cutting edge base has a radius between about 0.03 and 0.12 mm. The ceramic material may comprise zirconium oxide or zirconium oxide with additives. The composition may further comprise a dye such as patent blue.

There is also provided a composition comprising one or more hydrazine derivative(s) for use in the detection of smear layer(s) in healthy dentin tissue. The one or more hydrazine derivative(s) may be as described herein.

There is also provided a composition comprising one or more hydrazine derivative(s) for the manufacture of a detection agent for detection of smear layer(s) in healthy dentin tissue. The one or more hydrazine derivative(s) may be as described herein.

There is also provided a composition comprising one or more hydrazine derivative(s) for use in the detection of carious dentin tissue free from smear layer(s). The carious dentin tissue may have been generated by use of acid etching as known in the art or by use of Carisolv®. The one or more hydrazine derivative(s) may be as described herein.

There is also provided a composition comprising one or more hydrazine derivative(s) for the manufacture of a detection agent for detection of carious dentin tissue free from smear layer(s). The carious dentin tissue may have been generated by use of acid etching as known in the art or by use of Carisolv®. The one or more hydrazine derivative(s) may be as described herein.

There is also provided a method of detecting smear layer(s) in healthy dentin tissue, said method comprising the steps of:

(i) addition of a composition comprising one or more hydrazine derivative(s) onto the dentin tissue and
(ii) visual examination of the dentin tissue stained by the composition comprising the one or more hydrazine derivative(s) in step (b). The dentin tissue under examination may be treated with acid etching as known in the art or Carisolv® prior to step (i). The method may further comprise one or more rinsing steps, wherein rinsing is performed with water, brine or an aqueous pharmacologically acceptable solution.

There is also provided a method for detection of carious dentin tissue free of smear layer comprising the steps of:

(i) addition of a composition comprising one or more hydrazine derivative(s) onto the carious dentin tissue free of smear layer, and
(ii) visual examination of the dentin tissue stained by the composition comprising the one or more hydrazine derivative(s) in step (b). The dentin tissue under examination may be treated with acid etching as known in the art or Carisolv® prior to step (i). The method may further comprise one or more rinsing steps, wherein rinsing is performed with water, brine or an aqueous pharmacologically acceptable solution.

There is also provided a method for removal of carious dentin tissue free from smear layer(s) comprising the steps of:

(iii) addition of a composition comprising one or more hydrazine derivative(s) to the carious dentin tissue free from smear layer(s), and
(iv) subjecting the carious dentin tissue free from smear layer to drilling by a dental instrument such as a dental drill that provides treated dentin tissue free from staining in the method according to the first aspect.

The method may further comprise one or more rinsing steps, wherein rinsing is performed with water, brine or an aqueous pharmacologically acceptable solution. Cooling may be performed upon drilling with the dental drill.

In the methods described herein, the composition comprising one or more hydrazine derivative(s) may be a preparation containing an active, caries-dissolving two-component liquid in the form of a first active, caries-dissolving component and the a second component which reduces the aggressiveness of the active component to mucous membranes, a gel substance and one or more hydrazine derivatives as described herein. The preparation may be Carisolv®, PerioPlus® or Perisolv including one or more hydrazine derivatives described herein. The one or more hydrazine derivatives may be a compound of formula (I) such as a compound (Ia), (Ib), (Ic) and/or (Id).

The invention is illustrated, but not limited, by the following Examples.

### EXAMPLES

Chemicals used in following examples were purchased from Ultradent, Sigma, Aldrich and Invitrogen, respectively.

SEEK was purchased from Ultradent Products (USA), Lucifer Yellow, i.e. compound Ia above where M is K⁺ was purchased from Sigma. Acid red 1 was purchased from Aldrich.

Alexa Fluor® 594 sodium salt, Alexa 350 and 5-(((2-(carbohydrazino)methyl)thio)acetyl)aminofluorescein were purchased from Invitrogen.

The chemical structure for Acid red 1 is indicated below.

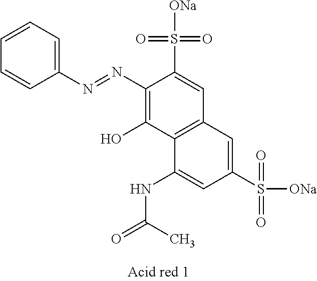

Patent blue was purchased from Sigma, Sweden.

### ABBREVIATIONS

### FTIR Fourier Transform Infrared Spectroscopy

TOF SIMS Time-of-flight secondary ionization mass spectrometry
FT Fourier transform

### IR Infrared Spectroscopy

u mass/charge
s second
nm nanometer
KeV Kilo electron Voltage
PEG polyethylene glycol
pA pico Ampere
amu atomic mass unite
MQ Utra pure water, from Milli-Q-systems, Milli Pore (USA)

### cP Centipoise

w/w weight of solute per weight of solution
M mol/liter
mM mmol/liter
microM micromol/liter
nm nanometer
rpm revolutions per minute

### Example 1

Two extracted permanent human teeth with no previous dental restorations were selected due to their severe carious tissue status and analysed with FTIR within one week after extraction. The outermost part of the carious dental lesions was removed. The remaining dental carious tissue was divided into two layers; one outer layer with discoloured, soft and infected dental carious tissue and one inner layer that was seen as uncoloured and excavated down to the estimated hardness of the remaining healthy dentin by tactile procedures. For each tooth, one sample was taken from healthy dentin and another sample was taken from the inner layer of carious tissue. After incubation in purified water they were left to dry at ambient temperature. The dry weight of each tooth sample was approximately 1 mg. Each sample was then mixed with potassium bromide (KBr) before subsequent FTIR examination with a total pellet weight of 100 mg. The IR analyses were performed using a Mattson Cygnus 100 FTIR spectrophotometer with 4 cm⁻¹ resolution. The instrument was purged with analytical instrument quality air to remove atmospheric CO₂ and H₂O, dried and purified with a Balstron type 75-60 conditioner. The spectra were baseline corrected using the FTIR software. For all spectra, the same wave-number positions were chosen. Each spectrum was acquired from 100 scans. For enhancing and further surveying peaks or specific shoulders, a Fourier Self-Deconvolution technique was used followed by spectral subtraction with sound healthy dentin set as reference.

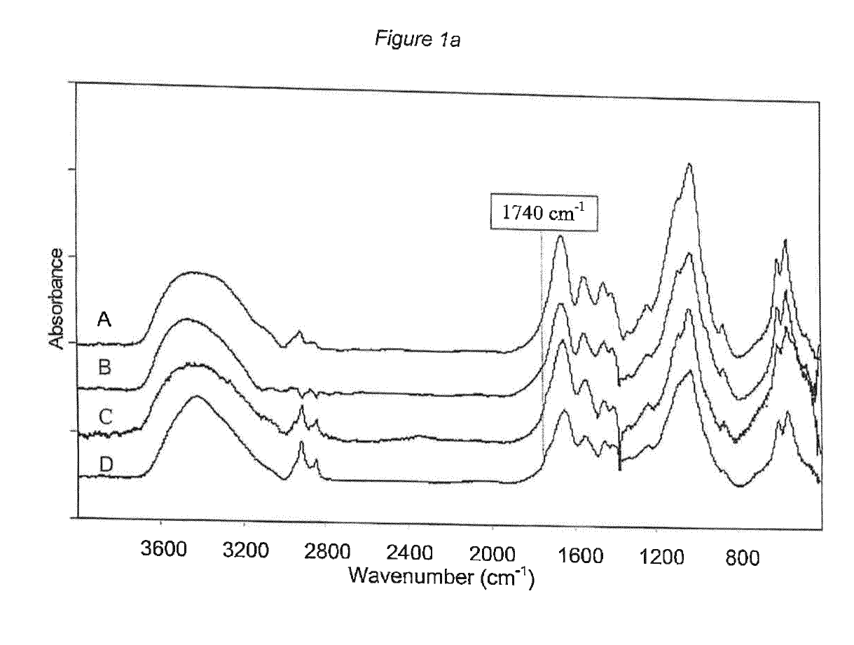

FIG. 1a shows the resulting deconvoluted FTIR spectra. Curves A and B result from samples taken from the healthy dentin tissue of the two extracted teeth. Curves C and D result from samples taken from the inner layer of carious tissue of the two extracted teeth. Close inspection shows that curves C and D exhibit a small peak (a “shoulder”) at 1740 cm⁻¹. This peak is lacking in curves A and B. The presence of a peak at 1740 cm⁻¹ in carious tissues has been reported earlier in WO 2008/048170, and has been attributed to the presence of ester groups in carious tissue.

FIG. 1b shows an FTIR spectrum where curve B has been subtracted from curve C and curve D in FIG. 1a resulting in curve C′ and curve D′. The peak at 1740 cm⁻¹ appears very clearly in curves C′ and D′. This is clear evidence that ester groups are only present in carious dentin tissue and not in healthy dentin tissue.

### Example 2

This experiment aimed to see if the added colours SEEK, Acid red 1, Lucifer Yellow, a combination of Lucifer Yellow and Acid red 1, or 5-(2-2-hydrazinyl-2-oxoethylthio)acetamido)-2-(3-hydroxy-6-oxo-6H-xanthen-9-yl)benzoic acid, Alexa Fluor® 594 and Alexa 350 bind to the surface of caries or not, after excavation down to a clinically caries free denoted surface. Experiments were performed during 4 days.

Whole caries infected tooth was added to an ortho acryl gel, which after hardening was used as a holder prior to sawing (saw of brand Zaw Micro Tone, German). Slices, having a thickness of 150 μm, were left over night in each colour to be tested (Day one). On the second day, the colours were washed out by rinsing the slices with MQ water and the teeth sections were photographed under either a microscope (6.7×; visible light), in UV (no microscope) or under a fluorescence microscope (100×). Thereafter, an aqueous salt solution (NaCl, 1 M) was added to the slices and incubated overnight. On the third day the slices were washed with MQ water and thereafter the same detection procedures were used as described above. Finally the tooth slices were exposed to an aqueous solution of NaOH (0.5 M) over night. The washing and detection path were then repeated as described previously (Day 4).

Inspection of the slices after incubation with 1 M NaCl (Day 2) showed that the slice treated with Acid red 1 was discoloured and only little staining remained. After washing with NaOH (Day 4) no colouring of the slice treated with Acid red 1 could be detected using visible light and barely with microscope. In contrast, slices treated with Lucifer Yellow, a combination of Lucifer Yellow and Acid red 1, 5-(2-2-hydrazinyl-2-oxoethylthio)acetamido)-2-(3-hydroxy-6-oxo-6H-xanthen-9-yl)benzoic acid, Alexa Fluor® 594 and Alexa 350 still contained colourants after day 4 as detected by fluorescence microscopy. Also, slices treated with SEEK remained stained though the colour seemed to be spread in the enamel, the healthy dentin and the root.

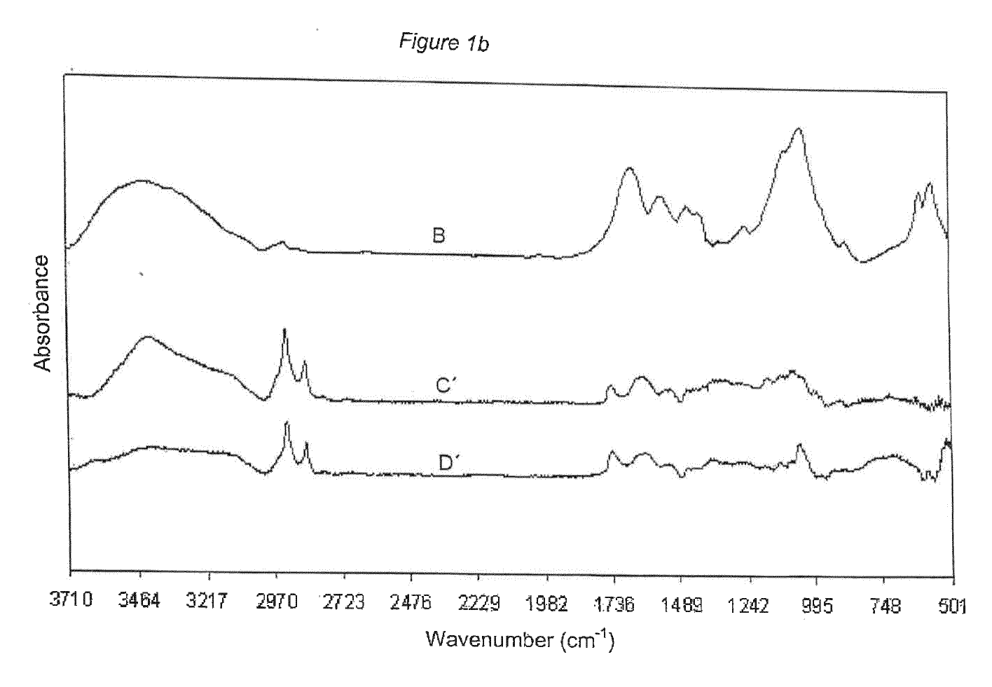

FIG. 2a shows staining of a tooth slice treated with 5-(2-2-hydrazinyl-2-oxoethylthio)acetamido)-2-(3-hydroxy-6-oxo-6H-xanthen-9-yl)benzoic acid after incubation with NaCl and NaOH.

FIG. 2b shows staining of a tooth slice treated with Lucifer Yellow sodium salt after treatment with NaCl and NaOH. The stained area is indicated with a circle and (1).

FIG. 2c shows staining of a tooth slice treated with Alexa Fluor 594 ® after treatment with NaCl and NaOH.

It was concluded that Acid red 1 binds in a reversible way to carious tissue in infected teeth, whereas 5-(2-2-hydrazinyl-2-oxoethylthio)acetamido)-2-(3-hydroxy-6-oxo-6H-xanthen-9-yl)benzoic acid, Lucifer Yellow, Alexa Fluor® and Alexa 350 bind irreversibly to carious dentin tissue in infected teeth. The reason why Acid red 1 binds reversibly may be that it is only capable of forming electrostatic bonds to carious tissue. SEEK binds in an unspecific way to carious tissue, healthy dentin and the root.

### Example 3

Extracted permanent human teeth with no previous dental restorations were selected due to their severe carious tissue status and analysed with FTIR within one week after extraction. The outermost part of the carious dental lesions was removed. The remaining dental carious tissue was divided into two layers; one outer layer with discoloured, soft and infected dental carious tissue and one inner layer that was excavated down to the observed uncoloured hard surface of the remaining healthy dentin controlled by tactile procedures.

Healthy dentin tissue and carious dentin tissue from the innermost layer were collected. This healthy dentin tissue sample and carious dentin tissue sample were then each divided into three samples.

The healthy dentin tissue sample (26 mg) was divided and treated as follows. Sample one, hereinafter denominated S1, (11 mg) was repeatedly washed in purified water and put under vacuum and defined as untreated sample, i.e. the reference sample. The second sample, hereinafter denominated sample S2, (8 mg) was rigorously washed with purified water. Thereafter sample S2 was mixed with an aqueous solution of the hydrazine derivate Lucifer Yellow (13 mM) that after reaction overnight was washed with water and salt (NaCl, 1 M). Fluorescence of bounded hydrazine derivate Lucifer Yellow was checked by a UV lamp. Next followed the addition of an aqueous solution of NaOH (0.5M), twice, to deprotonate the sample S2 and finally rinsed again with MQ water and dried over vacuum. The last step was used for determining whether or not the bonding was of electrostatic character. The third sample, hereinafter denominated sample S3, (7 mg) and the last procedure was treated with NaBH₄ (0.5 M in ethanol) and washed with concentrated ethanol to reduce aldehydes and ketones before the addition of an aqueous solution of the hydrazine derivate Lucifer Yellow (13 mM), repeated rinsing with purified water before dried and analysed.

The carious dentin tissue samples were treated in the same way as the healthy dentin sample above. The samples were denominated S4, S5 and S6. Sample S4 was subjected to the same treatment as sample S1 above. Sample S5 was subjected to the same treatment as sample S2 above. Sample S6 was subjected to the same treatment as sample S3 above.

Thus, in total 6 samples (three from the healthy dentin tissue and three from the carious dentin tissue) were analysed with FTIR within one week after extraction. The IR analyses were performed using a Nicolet 6700 FTIR spectrophotometer. A Smart Orbit diamond micro-ATR (Attenuated Total Reflectance) attachment was used to directly acquire spectra from the samples.

The instrument was purged with analytical instrument quality air to remove atmospheric CO₂ and H₂O, dried and purified with a Balstron type 75-60 conditioner.

The spectra were baseline corrected using the FTIR software. For all spectra, the same wavenumber positions were chosen. Each spectrum was acquired from 100 scans and the resolution was 4 cm⁻¹.

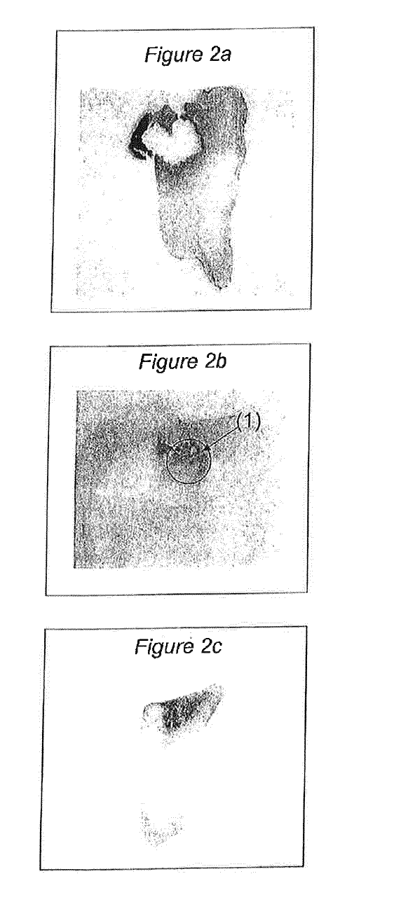

FIG. 3a shows the resulting FTIR spectrum for samples S1, S2 and S3 between 3700 and 2600 cm⁻¹. The same peaks appeared for S1, S2 and S3.

FIG. 3b shows the resulting FTIR spectrum for samples S4, S5 and S6 between 3700 and 2600 cm⁻¹. The same peaks appeared for S4, S5 and S6, different from the healthy dentin tissue samples in area of 2850 cm⁻¹.

FIG. 3c shows the resulting FTIR spectrum for samples S1, S2 and S3 between 1800 and 400 cm⁻¹. The curves obtained from S1, S2 and S3 exhibited no peak at 1740 cm⁻¹.

FIG. 3d shows the resulting FTIR spectrum for samples S4, S5 and S6 between 1800 and 400 cm⁻¹. The curve obtained from S4 exhibited a peak at 1740 cm⁻¹. The curves obtained for S5 and S6 exhibited no peak at 1740 cm⁻¹.

FIG. 3e shows an enhanced region of the FTIR spectra for samples S4, S5, S6 and S1 (dentin ref) at wave numbers 1800-1680 cm−1.

From FIGS. 3a and 3c it can be concluded that no reaction takes place between healthy dentin and the hydrazine derivative Lucifer Yellow. From FIG. 3d it can be concluded that reaction has taken place between the carious dentin tissue and the hydrazine derivative Lucifer Yellow, since the peak at 1740 cm⁻¹ is no longer present for samples S5 and S6. Accordingly, the hydrazine derivative Lucifer Yellow selectively reacts with carious dentin tissue.

### Example 4

FTIR-ATR and TOF-SIMS analyses were performed on a sample group consisting of carious tissue inner most layers selected and estimated with an exactness of a working dentist. Samples were pooled from 4-6 teeth of inner layer of carious tissue (37 mg). The pooled sample was divided into two subgroups where one was exposed to the hydrazine derivate Lucifer Yellow and further analysed with TOF-SIMS and the other only analysed with FTIR-ATR. An aqueous solution of the hydrazine derivate Lucifer Yellow (1.8 mM) was added to the inner layer carious tissue (19 mg) and after 1 hour exposure washed with an aqueous solution of NaOH (0.5 M) and purified water before dried over vacuum. Events prolonged under a couple of hours from extraction to excavation to the drying and milling of the substances. Both FTIR-ATR and TOF-SIMS followed. FTIR-ATR was performed as described in Example 3 above. Time-of-flight secondary mass spectrometry is a sensitive surface analysis that gives information of the molecular composition of the surface down to −1 nm in the material. The method is based on separations of charged secondary mass ions emitted from the sample surface after a pulsed beam of primary ions are projected to the sample holder. The primary ion used was 25 KeV Bi³⁺ with beam (current) at 0.12 pA. Each sample was attached to double side tape and analysed with a TOF-SIMS instrument (TOF-SIMS IV, CAMECA/IONTOF, GmbH, Germany). Positive secondary ion mass spectra were recorded from different areas of the sample at a size of 200×200 μm² and separated in a mass analyser and next compared with known mass spectra from the Ion Spec application (IonTof, GmbH, Germany, ver. 4.1) linked with the TOF-SIMS instrument. Negative secondary ion mass spectra were also recorded but gave no further information.

Data acquisition time for each secondary ion spectra spectrum was 100 s.

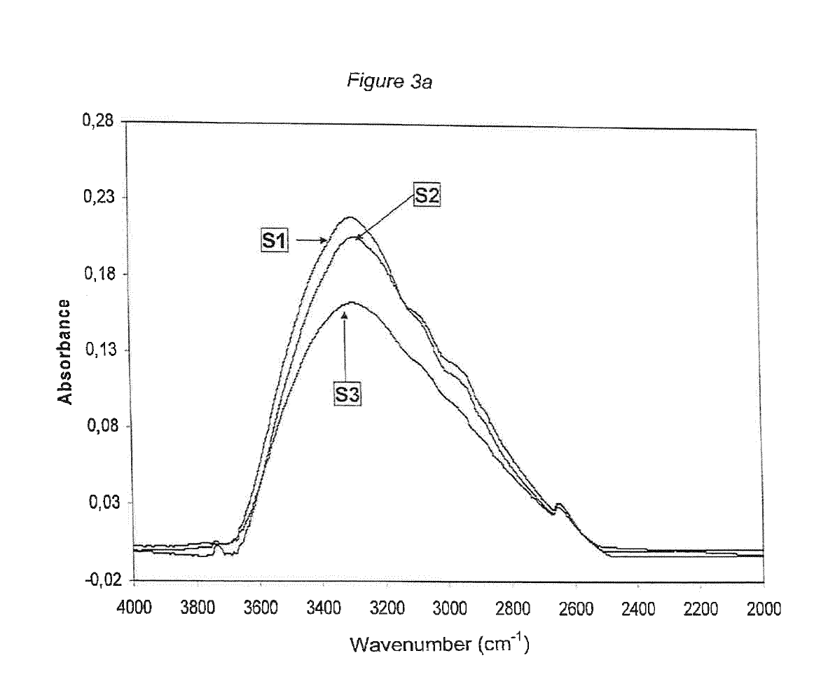

FIG. 4a shows positive TOF-SIMS spectra for carious dentin tissue. The upper spectrum was recorded for the sample of carious dentin tissue that had been milled but not subjected to treatment with hydrazine (i.e. the carious dentin tissue reference). The lower spectrum was recorded for the sample of carious dentin tissue that had been treated with the hydrazine derivative Lucifer Yellow.

FIG. 4b shows positive TOF-SIMS spectra for healthy dentin tissue. The upper spectrum was recorded for the sample of healthy dentin tissue that had been milled but not subjected to treatment with hydrazine (i.e. the healthy dentin tissue reference). The lower spectrum was recorded for the sample of healthy dentin tissue that had been treated with the hydrazine derivative Lucifer Yellow.

These spectra show that the carious dentin tissue reference holds the largest mass at 652.56 u, whereas the carious dentin tissue that had been treated with hydrazine Lucifer Yellow holds masses up to 1505.56 u. Lucifer Yellow has no masses higher than 600 u (not shown). It can therefore be concluded that masses higher than that seen for the carious dentin tissue reference sample at 652.56 u (FIG. 4a, upper spectrum) originate from a structure of carious dentin tissue covalently bonded to the hydrazine derivate Lucifer Yellow (FIG. 4a lower spectrum). The observed pattern of mass units repeatedly differing by 106 mass units corresponding to C5NO2 may be ascribed to Lucifer Yellow covalently bound to the carious tissue. This pattern was not detected for the healthy dentin samples. The positive mass spectra of the healthy dentin samples treated with Lucifer Yellow (FIG. 4b, lower spectrum) were similar to the positive mass spectra of untreated healthy dentin sample (FIG. 4b, upper spectrum). Both hold repeated masses with a difference of 56 mass units that correlates with CaO of the mineral. It was therefore concluded that Lucifer Yellow did not react with healthy dentin. Accordingly, the hydrazine derivative Lucifer Yellow selectively reacts with carious dentin tissue.

### Example 5

Carious dentin tissue of a pre molar tooth was treated with Carisolv® to obtain a cavity and subsequently stained with an aqueous solution of the hydrazine derivative Alexa 594. The concentration of the aqueous solution of the hydrazine derivative Alexa 594 was 15-50 mM. As a result, the cavity exhibited an intense dark bluish colour. Carisolv® was added to the cavity and excavation took place with a hand instrument from Mediteam Dental AB to reach a carious free level. Rinsing with MQ water was performed and a photograph was taken that showed a surface that appeared to be free of caries. A dentist performed tactile control of this surface and confirmed that the surface was free of caries.

In order to be absolutely certain that no caries remained further staining of the surface with Alexa 594 was attempted resulting in a very slight staining at the center of the cavity. The slightly stained surface was subjected to treatment with Carisolv® followed by excavation with a hand instrument. A final attempt to stain the thus obtained surface was made with Alexa 594, but no staining took place. It was therefore concluded that all carious dentin tissue of the tooth had been removed. It was concluded that a hydrazine derivative such as Alexa 594 can be used in combination with a chemical means for carious dentin tissue such as Carisolv® for selective detection and removal of carious dentin tissue. Further, it was concluded that staining with Alexa 594 is a better and more sensitive method for detecting carious dentin tissue than tactile control performed by a dentist.

### Example 6

Carious dentin tissue of a pre molar tooth stained with an aqueous solution of the hydrazine derivative Alexa 594 in a concentration of 15-50 mM was subjected to mechanical treatment by a dental drill until no staining could be visually detected. The dental drill was a high-speed dental drill Diamond/Fissure bur OB 301A FG from Plandent Forssbergs Dental, Sweden. An aqueous solution of the hydrazine derivative Alexa 594 in a concentration of 15-50 mM was added and resulted in staining. The stained surface was subjected to drilling until no staining could be visually detected. A dentist examined the surface visually and with a tactile instrument and concluded that the surface was free of caries. Surprisingly, addition of an aqueous solution of the hydrazine derivative Alexa 594 in a concentration of 15-50 mM to the surface free of caries resulted in staining. Again, drilling of the stained surface took place until no staining could be observed. Addition of an aqueous solution of the hydrazine derivative Alexa 594 in a concentration of 15-50 mM took place, and resulted in staining. Drilling and staining as described above in this example was repeated several times. The result was that staining always took place after the drilling had been performed. It was concluded that selective detection and removal of carious dentin tissue using a hydrazine derivative in combination with the dental drill of this Example did not work. It is suggested that the unsuccessful attempt to selectively detect and remove carious dentin tissue may be due to the formation of a smear layer. This is in contrast to the successful selective detection and removal of carious dentin tissue using a hydrazine derivative in combination with a chemical means for treatment of carious dentin tissue as described in Example 5.

### Example 7

An extracted tooth in a healthy state with no previous dental restoration was subjected to drilling with the dental drill Cera Bur at a speed of approximately 1500 rpm. The dental drill Cera Bur was from the company Comet USA LLC and is described in EP 1 849 429. However, the Cera Bur drill did not allow for boring through the healthy tooth enamel. Therefore, the tooth was subjected to a high-speed dental drill resulting in a cavity exposing dentin tissue, i.e. a dentin tooth surface. The high speed dental drill was the high-speed dental drill Diamond/Fissure bur OB 301A FG from Plandent Forssbergs Dental, Sweden. Addition of a mixture of Lucifer Yellow (15-50 mM) and patent blue (10-50 microM) in PEG followed by rinsing with water resulted in staining. The staining was visually detected using the instrument PoliLed SN06LP1121 operated at 460-470 nm, Faro. Since the tooth was known to be in a healthy state it was concluded that a smear layer was formed on the dentin tooth surface upon drilling with the high-speed dental drill, and that Lucifer Yellow stained this smear layer.

The thus formed smear layer stained with Lucifer Yellow was subjected to drilling with the dental drill Cera Bur at a speed of approximately 1500 rpm after which rinsing with water took place. Addition of a mixture of Lucifer Yellow (15-50 mM) and patent blue (10-50 microliters) in PEG followed by rinsing with water resulted in no staining. It was concluded that the Cera Bur drill removed the smear layer and the resulting dentin tissue was free of smear layer.

Addition of Carisolv® to the dentin tooth surface that had been subjected to drilling by the Cera Bur followed by rinsing with water and treatment with a mixture of Lucifer Yellow (15-50 mM) and patent blue (10-50 microM) in PEG resulted in no staining. It was concluded that the dentin tooth surface was free of smear layer. The thus obtained dentin tooth surface free of smear layer was subjected to drilling with the Cera Bur drill at a speed of approximately 1500 rpm followed by rinsing with water. Addition of a mixture of Lucifer Yellow (15-50 mM) and patent blue (10-50 microM) in PEG resulted in no staining. It was concluded that drilling with the Cera Bur drill did not yield smear layer(s).

### Example 8

An extracted tooth in a healthy state with no previous dental restoration was subjected to a high-speed dental drill resulting in a cavity exposing dentin tissue, i.e. a dentin tooth surface. The dental drill was a high-speed dental drill Diamond/Fissure bur OB 301A FG from Plandent Forssbergs Dental, Sweden. Addition of a mixture of Lucifer Yellow (15-50 mM) and patent blue (10-50 microM) in PEG followed by rinsing with water resulted in staining. The staining was visually detected using the instrument PoliLed SN06LP1121 operated at 460-470 nm, Faro. Since the tooth was known to be in a healthy state it was concluded that a smear layer was formed upon drilling, and that Lucifer Yellow stained this smear layer.

The thus formed smear layer stained with Lucifer Yellow was subjected to drilling with a round type steel dental drill 0210017/328457 from RA FFDM-Pneumat, France, at a speed of 1500 rpm after which rinsing with water took place. Addition of an aqueous solution of Lucifer Yellow in a concentration of 15-50 mM followed by rinsing with water resulted in staining. It was concluded that the dental drill of round type did not remove the smear layer.

Addition of Carisolv® followed by rinsing with water and addition of a mixture of Lucifer Yellow (15-50 mM) and patent blue (10-50 microM) in PEG resulted in no staining in the places that had been subjected to Carisolv®. It was concluded that the tooth surface was free of smear layer, and that Carisolv® had removed the smear layer. The thus obtained tooth surface free of smear layer was subjected to drilling with the round type steel at a speed of approximately 1500 rpm followed by rinsing with water. Addition of a mixture of Lucifer Yellow (15-50 mM) and patent blue (10-50 microM) in PEG resulted in staining as detected with the PoliLed instrument. It was concluded that drilling with the round type steel dental drill did yield a smear layer.

1. A method for testing a dental instrument comprising the steps of:
a) subjecting healthy dentin tissue free from smear layer(s) to treatment with a dental instrument, thereby yielding treated dentin tissue,
b) addition of a composition comprising one or more hydrazine derivative(s) to the treated dentin tissue obtained in step a),
c) examining if the treated dentin tissue in step b) is stained by the one or more hydrazine derivative(s), and
d) determining whether or not the treated dentin tissue contains a smear layer, wherein a stained dentin tissue in step c) indicates the presence of a smear layer, and wherein a lack of stained dentin tissue in step c) indicates the absence of a smear layer,
wherein said one or more hydrazine derivatives is a compound of formula (I)
RNHNH₂ (I)
wherein R is a chemical group containing a chromophore or forming a chromophore with NHNH₂.

2. A method according to claim 1, wherein the healthy tooth with dentin tissue free from smear layer(s) is obtained by subjecting the healthy tooth and the dentin tissue thereof to acid etching or to treatment with a preparation containing an active, caries-dissolving two-component liquid in the form of a first active, caries-dissolving component and the a second component which reduces the aggressiveness of the active component to mucous membranes, and a gel substance.

3. A method according to claim 2, wherein the preparation is an aqueous composition comprising a first active component NaOCl in a concentration of 1-2% (w/w), a second component having a pH between 9.5 and 10.5 and comprising a mixture of glutamic acid, leucine and lysine in a concentration of 0.5-1.5% (w/w), NaCl in a concentration of 0.5% (w/w), and high viscosity carboxymethyl cellulose gel in a concentration of 2.5-5% (w/w).

4. A method according to claim 2 or 3, wherein the preparation is removed from the tooth by a fluid flow, such as an air flow, and/or by a powder-like blasting, wherein a healthy tooth with dentin tissue free from smear layer(s) is obtained.

5. A method according to any one of claims 1 to 4, wherein the one or more compounds being a hydrazine derivative is selected from the group consisting of: 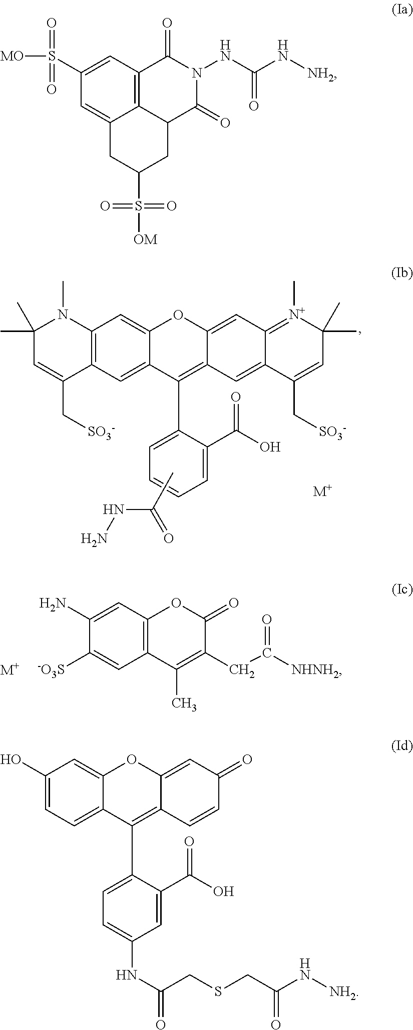
wherein M or M⁺ represents a monovalent metal ion selected from Li⁺, K⁺ and Na⁺, and

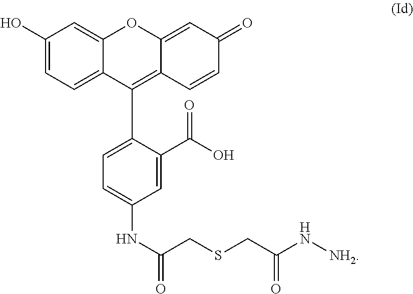 [SMILES: NNC(=O)CSCC(=O)Nc1ccc(-c2c3ccc(=O)cc-3oc3cc(O)ccc23)c(C(=O)O)c1]

6. A method according to any one of the previous claims, wherein the examination in step c) takes place using an instrument operating between 10 and 500 nm, preferably between 200 and 500 nm, and more preferably between 10 and 400 nm.

7. A method according to any one of the previous claims, wherein the dental instrument is a dental drill, and wherein the treatment with the dental instrument in step a) is provided by drilling with the dental drill thereby yielding drilled dentin tissue.

8. A method according to any one of claims 1 to 6, wherein the dental instrument is a root canal instrument, and wherein the treatment with the dental instrument in step a) is provided by treating a root canal of the healthy tooth with said root canal instrument, thereby yielding a treated root canal.

9. A kit of parts for detection and/or removal of carious dentin tissue comprising:
(i) a composition comprising one or more hydrazine derivatives of formula (I)
RNHNH₂ (I)
wherein R is a chemical group containing a chromophore or forming a chromophore with NHNH₂, and
(ii) a dental instrument that provides treated dentin tissue free from staining in the method according to any one of claims 1 to 8.

10. A kit of parts according to claim 9, wherein the one or more hydrazine derivatives is/are as defined in claim 5.

11. A kit of parts according to claim 9 or 10, wherein the dental instrument is a dental drill that provides drilled dentin tissue free from staining in the method according to claim 7.

12. A kit of parts according to claim 11, wherein the dental drill comprises a head having a rounded basic shape, and at least two cutting edges at a front side that merge into a transitional cutting edge, said dental drill comprising plastic having a hardness between 60 and 250 Knoop.

13. A kit of parts according to claim 11, wherein the dental drill comprises a head having a plurality of cutting edges, the head further having an at least rounded basic shape, wherein two of the cutting edges merge into a transitional cutting edge at a front side, and wherein the dental drill is made of a ceramic material and a cutting edge chipping is between about 2.5 and 7 μm and a cutting edge base has a radius between about 0.03 and 0.12 mm.

14. A kit of parts according to claim 9 or 10, wherein the dental instrument is a root canal instrument that provides treated dentin tissue free from staining in the method according to claim 8.

15. A kit of parts according to any one of claims 9 to 14, wherein the kit further comprises iii) a preparation containing an active, caries-dissolving two-component liquid in the form of a first active, caries-dissolving component and the a second component which reduces the aggressiveness of the active component to mucous membranes, and a gel substance for use in removal of smear layer(s) from tooth enamel or dentin tissue.

16. A kit of parts according to claim 15, wherein the preparation is an aqueous composition comprising a first active component NaOCl in a concentration of 1-2% (w/w), a second component having a pH between 9.5 and 10.5 and comprising a mixture of glutamic acid, leucine and lysine in a concentration of 0.5-1.5% (w/w), NaCl in a concentration of 0.5% (w/w), and high viscosity carboxymethyl cellulose gel in a concentration of 2.5-5% (w/w).

17. A kit of parts according to any one of claims 9 to 16, comprising a flow apparatus for providing a fluid flow, such as an air flow, or a powder-like blasting to a tooth.

18. A kit of parts according to any one of claims 9 to 17, further comprising instructions for detecting and/or removal of carious dentin tissue.

19. A method for detection and removal of carious dentin tissue, comprising the steps of:
if the carious dentin tissue comprises a smear layer(s), removing the smear layer(s) from the area of the carious dentin tissue
subjecting the carious dentin tissue of a tooth to treatment by an dental instrument, thereby yielding treated dentin tissue,
addition of a composition comprising one or more hydrazine derivative(s) to the dentin tissue,
examining if the treated dentin tissue free from a smear layer (s) is stained by the one or more hydrazine derivative(s), and
determining whether or not the treated tissue free from the smear layer (s) contains a carious dentin tissue, wherein a stained dentin tissue by said one or more hydrazine derivative(s) indicates the presence of carious dentin tissue, and wherein a lack of stained dentin tissue by said one or more hydrazine derivative(s) indicates the absence of carious dentin tissue
wherein said one or more hydrazine derivatives is a compound of formula (I)
RNHNH₂ (I)
wherein R is a chemical group containing a chromophore or forming a chromophore with NHNH₂.

20. A method according to claim 19, wherein said one or more hydrazine derivatives is/are as defined in claim 5.

21. A method according to claim 19 or 20, wherein the dental instrument provides a treated dentin tissue free from staining in the method according to any one of claims 1 to 8.

22. A method according to any one of claims 19 to 21, wherein the dental instrument is a dental drill, and wherein the treatment with the dental instrument is provided by drilling with the dental drill, thereby yielding drilled dentin tissue.

23. A method according to claim 22, wherein the dental drill comprises a head having a rounded basic shape, and at least two cutting edges at a front side that merge into a transitional cutting edge, said dental drill comprising plastic having a hardness between 60 and 250 Knoop.

24. A method according to claim 22, wherein the dental drill comprises a head having a plurality of cutting edges, the head further having an at least rounded basic shape, wherein two of the cutting edges merge into a transitional cutting edge at a front side, and wherein the dental drill is made of a ceramic material and a cutting edge chipping is between about 2.5 and 7 μm and a cutting edge base has a radius between about 0.03 and 0.12 mm.

25. A method according to any one of claims 19 to 21, wherein the dental instrument is a root canal instrument, and wherein the treatment with the dental instrument is provided by treating root canal of the healthy tooth with said root canal instrument, thereby yielding a treated root canal.

26. A method according to any one of claims 19 to 25, wherein the treatment by the dental instrument provides for the removal of the smear layer that may be present in the carious dentin tissue.

27. A method according to any one of claims 19 to 26, further comprising adding a preparation containing an active, caries-dissolving two-component liquid in the form of a first active, caries-dissolving component and the a second component which reduces the aggressiveness of the active component to mucous membranes, and a gel substance for use in removal of smear layer(s) from tooth enamel or dentin tissue.

28. A method according to claim 27, wherein the preparation is an aqueous composition comprising a first active component NaOCl in a concentration of 1-2% (w/w), a second component having a pH between 9.5 and 10.5 and comprising a mixture of glutamic acid, leucine and lysine in a concentration of 0.5-1.5% (w/w), NaCl in a concentration of 0.5% (w/w), and high viscosity carboxymethyl cellulose gel in a concentration of 2.5-5% (w/w).

29. A method according to claim 27 or 28, wherein the preparation is removed by a fluid flow, such as an air flow, and/or by a powder-like blasting, wherein carious dentin is removed.

30. A method according to claim 27, 28 or 29, wherein the preparation includes said one or more hydrazine derivatives of formula (I).
